(19) United States
(12) Patent Application Publication
(10) Pub. No.: US 2024/0173397 A1
(43) Pub. Date: May 30, 2024

(54) LIMITING VIRAL INFECTION AND REPLICATION IN STEM CELLS

(71) Applicant: GEORGE MASON UNIVERSITY, Fairfax, VA (US)

(72) Inventors: Yuntao Wu, Fairfax, VA (US); Sijia He, Fairfax, VA (US); Deemah Dabbagh, Fairfax, VA (US)

(73) Assignee: GEORGE MASON UNIVERSITY, Fairfax, VA (US)

(21) Appl. No.: 18/522,524

(22) Filed: Nov. 29, 2023

Related U.S. Application Data
(60) Provisional application No. 63/428,533, filed on Nov. 29, 2022.

(51) Int. Cl. A61K 39/21; A61K 39/00; A61K 39/145; A61K 39/39; A61P 31/18; C07K 14/705; C12N 7/00; C12N 15/86

(52) U.S. Cl. CPC A61K 39/21; A61K 39/145; A61K 39/39; A61P 31/18; C07K 14/70596; C12N 7/00; C12N 15/86; A61K 2039/5256; A61K 2039/55516; C12N 2740/13043; C12N 2740/13051; C12N 2740/16043; C12N 2740/16051; C12N 2760/16143; C12N 2760/16151

(57) ABSTRACT

An Embodiment relates to a method comprising: co-expressing a viral vector of a virus of interest and a CD34 expression vector into a human cell line to produce virus particles such that CD34 incorporates into the virus particles; isolating the virus particles from the human cell line; blocking a viral infection; and wherein the method is configured for inhibiting the viral infection of the virus of interest.

### RELATED APPLICATION

This application claims priority from U.S. provisional application 63/428,533 titled as “Limiting Viral Infection and Replication in Stem Cells” filed on Nov. 29, 2022, which is incorporated by reference in its entirety.

This application also relates to U.S. Provisional Patent Application No. 62/828,381, entitled “THE USE OF PSGL-1 (P-SELECTIN GLYCOPROTEIN LIGAND-1) TO INACTIVATE ALL ENVELOPED VIRUSES FOR PRODUCING LIVE-ATTENUATED VACCINES,” filed Apr. 2, 2019, the disclosure of which is hereby incorporated by reference herein in its entirety. This application is related to U.S. patent application Ser. No. 16/271,100, filed Feb. 8, 2019, entitled “METHOD AND SYSTEM FOR INACTIVATING VIRUS INFECTIVITY FOR PRODUCING LIVE-ATTENUATED VACCINES,” which is incorporated by reference herein in its entirety.

### FIELD OF THE INVENTION

The Invention relates generally to method of blocking viral infectivity to target cells, more specifically to the method of inhibiting RNA virus replication through expression of CD34 protein.

### BACKGROUND OF INVENTION

CD34 is also a member of the sialomucin family of proteins, and was found to bind to L-selectin, P-selectin, and E-selectins (11, 12). A ligand for these selectins, PSGL-1, has recently been identified as a broad-spectrum antiviral restriction factor, inactivating virus infectivity through virion incorporation that inhibits virion attachment to target cells (13-15).

CD34 is a transmembrane phosphoglycoprotein originally identified on hematopoietic stem cells (1, 2) and on the progenitor cells of a variety of tissues (3). Expression of CD34 is generally associated with the phenotypes of hematopoietic cells and multiple nonhematopoietic progenitor cells, and thus serves as a common marker of diverse progenitor cells (3).

Nevertheless, little is known about the exact function of CD34, although the molecule has been suggested to facilitate cell adhesion and migration (4-8) or, under some circumstances, block cell adhesion (9, 10).

### SUMMARY OF INVENTION

In an embodiment, we investigated whether CD34 has similar antiviral activity, using HIV-1 and MLV infection as a model.

In an embodiment, results demonstrate that expression of CD34 inhibits retrovirus replication in CD4 T cells and undifferentiated CD34+ lymphoblastic progenitor cells. Mechanistically, surface expression of CD34 on target cells leads to its incorporation into progeny virions released from infected CD34+ cells, and this CD34 incorporation inactivates virus infectivity through inhibition of particle attachment to new target cells. These results suggest that the ubiquitous expression of CD34 on progenitor cells may serve as a host innate immune defense limiting viral dissemination in these critical cell populations.

In an embodiment, an in vitro method comprising: co-expressing a viral vector of a virus of interest and a CD34 expression vector into a human cell line to produce virus particles such that CD34 incorporates into the virus particles; isolating the virus particles from the human cell line; blocking a viral infection; and wherein the method is configured for inhibiting the viral infection of the virus of interest.

In an embodiment, wherein the virus particles comprise non-infectious virus particles, attenuated virus particles or inactivated virus particles of the virus of interest.

In an embodiment, wherein the expression vector is in an amount of about 0.5 ng to about 400 ng.

In an embodiment, wherein a ratio of the CD34 expression vector and the viral vector is about 1:1 to 1:1000.

In an embodiment, wherein the CD34 expression vector inhibits the viral infection in a dose dependent manner.

In an embodiment, wherein the virus of interest comprises HIV or MLV.

In an embodiment, wherein the CD34 is configured to sterically hinder the virus particles attachment to its corresponding target cells.

In an embodiment, wherein the CD34 inhibits attachment of the viral particles to the target cells in an envelope glycoprotein-independent manner.

In an embodiment, wherein the viral particles are virions.

In an embodiment, wherein the method is configured to reduce the viral infection by about 25% to about 85%.

An embodiment relates to a composition comprising a recombinant virus particle comprising an overexpressed CD34 protein; wherein the recombinant virus particle comprises a non-infectious virus particle, an attenuated virus particle or an inactivated virus particle or a combination thereof.

In an embodiment, the virus comprising the overexpressed CD34 protein comprises more than one copy of CD34 protein.

In an embodiment, wherein the composition is configured to treat a viral infection.

In an embodiment, wherein the viral infection is caused by an enveloped virus.

In an embodiment, wherein the enveloped virus comprises RNA virus.

In an embodiment, wherein the composition is configured to reduce a viral infection by about 25% to about 95%.

In an embodiment, wherein the composition comprises a pharmaceutically acceptable excipient.

In an embodiment, wherein the composition comprises an effective amount of the recombinant virus to treat a viral infection.

In an embodiment, wherein the composition comprises an adjuvant comprising an interleukin.

In an embodiment, wherein the enveloped virus comprises HIV, MLV or influenza.

An embodiment, an ex vivo method comprising taking CD34+ cells; transfecting a viral vector expressing a virus of interest into the CD34+ cells; producing viral particles in the CD34+ cells and isolating the virus particles from the CD34+ cells, and wherein the viral particle are non-infectious virus particles, attenuated virus particles or inactivated virus particles of the virus of interest.

In an embodiment, wherein the viral particles are configured to inhibit a viral infection of the virus of interest.

In an embodiment, wherein the viral infection comprises HIV.

In an embodiment, wherein the viral particles are configured to be used as a vaccine.

In an embodiment, wherein inactivation of the virus particles infectivity is independent of an envelope glycoprotein.

In an embodiment, wherein the viral infection an influenza virus.

In an embodiment, wherein the viral infection comprises MLV virus.

In an embodiment, wherein the virus particles comprise non-infectious virus particles.

In an embodiment, wherein the virus particles comprise attenuated virus particles.

In an embodiment, the viral particles are configured as a personalized medication for treatment of a viral infection.

In an embodiment, a method is configured to produce viral particles configured to inhibit the viral infection.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1: Dosage-dependent inactivation of HIV-1 virion infectivity by CD34. (A) Schematic of virion production in the presence of CD34 in virus producer cells. (B) Effects of CD34 on HIV-1 viral release. HEK293T cells were cotransfected with HIV(NL4-3) DNA (1 μg) plus a CD34-expression vector (0.5 to 400 ng of DNA). Virion release was quantified at 48 h post cotransfection by HIV-1 p24 ELISA. Data are represented as mean±SD from ELISA triplicate. (C to E) CD34 inactivates HIV-1 virion infectivity. HEK293T cells were cotransfected with HIV(NL4-3) DNA (1 μg) plus a CD34-expressing vector (0.5 to 400 ng of DNA). Virions were harvested at 48 h and normalized for p24, and viral infectivity was quantified by infecting Rev-A3R5-GFP indicator cells. Shown are the percentages of GFP+ cells at 48 h post infection. The experiment was repeated more than 3 times. In all co-transfection experiments, an empty vector (Vector) was used to ensure an equal amount of total plasmid DNA was used in all co-transfection.

FIG. 2: Virion incorporation of CD34 and its effects on Env incorporation and virion attachment to target cells. (A to C) Virion incorporation of CD34. HEK293T cells were cotransfected with 10 μg HIV-1(NL4-3) plus 2 μg of CD34 DNA (HIV+CD34) or with CD34 DNA only (CD34 only). Supernatants were harvested at 48 h, filtered, concentrated, and purified by ultra-speed centrifugation through a 6%-18% OptiPrep gradient. CD34 and viral p24 proteins in each fraction were analyzed by western blot using antibodies against HIV-1 p24 or CD34. Shown is the representative of 3 repeats. Representative blots from 3 independent assay repeats are shown. (D and E) Schematic of the immunomagnetic capture assay used to detect CD34 proteins on HIV-1 particles (D). HEK293T cells were cotransfected with HIV-1(NL4-3) DNA (1 μg) plus various amounts of a CD34 expression vector (0.5-10 ng) or an empty vector DNA. Supernatants were harvested at 48 h and incubated with magnetic beads coated with antibody against CD34 or an isotype control antibody. Captured particles were washed, eluted, and quantified for the p24 levels (E). p-values were calculated based on 3 independent capture assays. (F) Effects of CD34 on HIV-1 Env incorporation. HEK293T cells were cotransfected with HIV(NL4-3) DNA plus an CD34 expression vector (10 and 50 ng) or an empty vector. Particles were harvested at 48 h and purified through a sucrose gradient. Virions were lysed and analyzed with western blot using antibodies against HIV-1 gp41 and p24. Representative blots from 3 independent experiment repeats are shown. (G) CD34 blocks HIV-1 virion attachment to target cells. Viral particles were produced by cotransfection of HEK293T cells with HIV-1(NL4-3) DNA (1 μg) and a CD34 expression vector or an empty vector (10 ng). HIV-1 p24-normalized viral particles were then assayed for attachment to target Rev-A3R5-GFP cells by western blot for cell-bound p24. Representative blots from 3 independent assays are shown. The protein band intensity was quantified using NIH ImageJ and the relative ratios of vector/GAPDH and CD34/GAPDH were calculated (blue and red dots). Vector/GAPDH ratios were assigned as “1”. The averages (blue and red lines) and p-values are shown. p-values were calculated using two-tailed T-test. (H) CD34 blocks HIV-1 virion attachment independent of Env interaction with cellular receptors. Viral particles were produced by similarly by cotransfection of HEK293T cells with HIV-1(NL4-3) DNA or HIV-1(NL4-3/KFS) and a CD34 expression vector or an empty vector. HIV-1 p24-normalized viral particles were then assayed for attachment to target HeLa or HeLa JC.53 cells by western blot for cell-bound p24. Representative blots from 3 independent assays are shown. The protein band intensity was quantified using NIH ImageJ and the relative ratios of vector/GAPDH and CD34/GAPDH were calculated (blue and red dots). Vector/GAPDH ratios were assigned as “1”. The averages (blue and red lines) and P values are shown. p-values were calculated using two-tailed T-test.

FIG. 3: Expression of CD34 in human CD4 T cells inhibits HIV infection. (A) Schematic of the assay to determine effects of CD34 expression in human CD4 T cells. (B) Quantification of surface CD34 expression on human CD4 T cells, Rev-A3R5-GFP, following electroporation of cells with a CD34 expression vector or an control empty vector. (C and D) Expression of CD34 in human CD4 T cells, Rev-A3R5-GFP, inhibits HIV infection. Cells were electroporated with a CD34-expression vector or a control empty vector. At 48 h post electroporation, cells were infected with HIV-1(NL-4-3). HIV-1 replication was quantified by Rev-dependent expression of GFP at 3 days post infection (C). Cells were also similarly electroporated with DNA, and then infected with a single-cycle replication virus, HIV-1(KFS)(Env). HIV-1 infection was quantified by GFP expression at 3 days post infection (D). The experiments were independently repeated 3 times. For statistical analysis, the infection rates (% GFP⁺ cells) of empty vector-electroporated cells, infected with HIV-1- or HIV-1(KFS)(Env), were assigned as “1”. The averages (blue and red lines) and p-values are shown. p-values were calculated using two-tailed T-test. (E) Effects of CD34 on HIV-1 viral entry. Rev-A3R5-GFP CD 4 T cells were electroporated with a CD34-expression vector or a control empty vector. At 48 h post electroporation, cells were infected with HIV-1(NL-4-3) for 2 hours. Equal p24 was used for the assay. Infected cells were used for viral entry assay (BlaM). The percentages of cells with cleaved CCF2 are shown. The experiments were independently repeated 4 times. The averages of the percentage of cells with cleaved CCF2 (blue and red lines) and p-values are shown. p-values were calculated using two-tailed T-test.

FIG. 4: CD34 expression on Kasumi-3 cells and effects on HIV-1 infection. (A) Measurement of surface CD34 expression on Kasumi-3 cells and on primary human bone marrow CD34⁺ cells. Cell were stained with an antibody against CD34 or an isotype control antibody, and then analyzed by flow cytometry. (B) Measurement of surface CD4 and CXCR4 expression on Kasumi-3 cells. Cell were stained with antibodies against CD4 or CXCR4, and then analyzed by flow cytometry. (C) HIV-1 infection of Kasumi-3 cells. Cells were infected with HIV-1(89.6) for 4 h, washed, and then cultured for 6 days. Viral p24 release was quantified by ELISA using cell culture supernatant. Shown are ELISA triplicates of samples from one infection assay. The infection assays were repeated 3 times. (D and E) Down regulation of CD34 by GM-CSF plus TNF-□. Cells were cultured in GM-CSF plus TNF-□ or medium for 7 days, and surface expression of CD34 was measured by surface staining with an antibody against CD34 and then analyzed by flow cytometry. Shown are histoplot (D) and density plot (E) of CD34 expression. (F) Schematic of the assays performed in (G) to (I) to determine effects of CD34 on HIV infection of Kasumi-3 cell. Cells were cultured in GM-CSF plus TNF-□□ or medium for 7 days, and then electroporated with HIV-1 DNA or with HIV-1 DNA plus a CD34-expression vector. Viral particles were harvested at 3 days post electroporation, and virus infectivity was quantified by infection of Rev-A3R5-GFP indicator cells using equal levels of p24. (G) Culturing Kasumi-3 cells in GM-CSF plus TNF-□□ did not enhance viral release. Cells were cultured in GM-CSF plus TNF-□ and then electroporated with HIV-1 DNA. Virus release was quantified at 4 days post electroporation with p24 ELISA. (H) Culturing Kasumi-3 cells in GM-CSF plus TNF-□□ enhances HIV-1 infectivity. Cells were cultured in GM-CSF plus TNF-□ and then electroporated with HIV-1 DNA. Virus infectivity was quantified by infection of Rev-A3R5-GFP indicator cells using equal levels of p24. (I) Expression of CD34 in Kasumi-3 cells diminishes GM-CSF/TNF-□-mediated enhancement of HIV-1 infectivity. Cells were cultured in GM-CSF plus TNF-□ and then electroporated with HIV-1 DNA plus a CD34-expression vector. Virus infectivity was quantified by infection of Rev-A3R5-GFP indicator cells using equal levels of p24.

FIG. 5: Expression of multiple SHREK proteins on Kasumi-3 cells. Kasumi-3 cells were stained with antibodies against CD34, PSGL-1, CD43, CD164, PODX-1, PODXL-2, TMEM, TIM-1, MUC1, or MUC4 and then analyzed by flow cytometry. Kasumi-3 cells expressed CD34, PSGL-1, CD43, and CD164, but not PODXL-1, PODXL-2, TMEM, TIM-1, MUC1, or MUC4.

FIG. 6: HIV-1 infection of Kasumi-3 cells. (A) Cells were infected with HIV-1(89.6) for 3.5 h, washed, and then cultured for 10 days. Viral p24 release was quantified by ELISA using cell culture supernatant. (B) Kasumi-3 cells were electroporated with HIV-1(NL4-3) DNA, HIV-1(89.6) DNA, or a control empty vector (4 μg). Viral replication was quantified by p24 release using ELISA of the cell culture supernatant.

FIG. 7: GM-CSF/TNF-□-mediated downregulation of CD34 but not PSGL-1 and CD43. Kasumi-3 cells were cultured in GM-CSF plus TNF-□ or medium for 10 days, and surface expression of CD34, PSGL-1, and CD43 was measured by surface staining with antibodies against CD34, PSGL-1, or CD43 and then analyzed by flow cytometry. Shown are density plots of CD34, PSGL-1, and CD43 expression on Kasumi-3 cells cultured with or without GM-CSF plus TNF-□.

FIG. 8: CD34 restricts MLV virus infectivity. (A) Schematic of the assay performed to determine effects of CD34 on MLV infectivity. (B and C) HEK293T cells were cotransfected with pCL-Eco and pRetroQ-AcGFP-N1 plus a CD34 expression vector or an empty control vector. Following cotransfection, expression of CD34 in cells was validated by surface staining of CD34 and flow cytometry (B) or by western blot (C). (D) MLV virions were harvested, and viral infectivity was quantified by infecting NIH 3T3 cells and measuring GFP expression.

FIG. 9: HIV-1 and viral Vpu, Nef, Gag proteins do not downregulate CD34. (A) Kasumi-3 cells were electroporated with HIV-1(NL4-3) DNA, HIV-1(89.6) DNA, or a control empty vector (4 μg). Downregulation of surface CD34 was quantified at 4 and 7 days post electroporation. (B) HEK293T cells were transfected with a control empty vector or a CD34 expression vector (100 ng). Cells were also cotransfected with a CD34 expression vector plus HIV-1(NL4-3) DNA, a Gag expression vector, a Vpu expression vector, or a Nef expression vector (2 μg of each vector). Surface CD34 expression was quantified with flow cytometry at 48 h post-cotransfection.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

All numerical designations, e.g., pH, temperature, time, concentration, and molecular weight, including ranges, are approximations which are varied (+) or (−) by increments of 1.0 or 0.1, as appropriate, or, alternatively, by a variation of +1-15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art.

The following terms and phrases, unless otherwise indicated, shall be understood to have the following meanings.

“CD4 (cluster of differentiation 4)” is a glycoprotein found on the surface of immune cells such as T helper cells, monocytes, macrophages, and dendritic cells.

“Vector,” “expression vector,” or “construct” is a nucleic acid used to introduce heterologous nucleic acids into a cell, for example by a process of transfection, infection, or transformation that has regulatory elements to provide expression of the heterologous nucleic acids in the cell. Vectors include but are not limited to plasmid, minicircles, yeast, and viral genomes. In an embodiment, an expression vector comprises sufficient cis-acting elements for expression; other elements for expression can be supplied by the host cell or in an in vitro expression system.

In some embodiments, the vectors are plasmid, minicircles, yeast, or viral genomes. In some embodiments, the vector is a viral vector.

The term, “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

The term “expression” as used herein refers to the transcription and/or translation of a particular nucleotide sequence driven by an endogenous or exogenous promoter.

“Protein expression” refers to refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand.

The term “recombinant expression system” or “recombinant vector” refers to a genetic construct or constructs for the expression of certain genetic material formed by recombination. The term “recombinant” or “engineered” when used with reference, for example, to a cell, a nucleic acid, a protein, or a vector, indicates that the cell, nucleic acid, protein, or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein, or nucleic acid sequence, including of one or more amino acids, deoxyribonucleotides, or ribonucleotides; addition, deletion, or substitution of one or more of amino acids in the peptide or protein; or addition, deletion, or substitution of one or more of nucleic acids in the nucleic acid sequence.

The term “virus particle or virion particle” as used herein refers to a complete infectious agent that consists of an RNA or DNA core with a protein coat sometimes with external envelopes and that is the extracellular infective form of a virus. In an embodiment, virus could be a retrovirus.

The term “non-infectious virus particle” as used herein refers to a virus particle that has been rendered unable to infect a host cell and cause one or more effects, such as disease or death of the host cell.

The term “attenuated virus particle” as used herein refers to a virus particle that has been weakened in its ability to infect a host cell and cause one or more effects, such as disease or death of the host cell.

The term “inactivated virus particle” as used herein refers to a virus particle that has been rendered unable to infect a host cell and cause one or more effects, such as disease or death of the host cell.

The term “in vitro” as used herein refers to system established using cell lines in a culture laboratory.

The term “ex vivo” as used herein refers to cells that have been removed from a living organism, (e.g., a human) and propagated outside the organism (e.g., in a culture dish, test tube, or bioreactor).

The term “expressing or overexpressing CD34” in virus producing cells as used herein refers to causing the protein CD34 to be synthesized by the cell in a quantity larger than would normally be expressed by the cell on its own.

The term “immortalized human cell line” or “human cell line” means human cells that are not primary cells taken directly from an organism. In particular, it means a permanently established cell line that grows indefinitely as long as there is suitable fresh medium and space and deviates from the Hayflick limit.

In an embodiment, cell line suitable for the method of the present invention is selected from the group consisting of kidney, bladder, liver, lung, myocardium, smooth muscle, ovary or gastrointestinal cells. In an embodiment, immortalized cell lines containing the vector are cultured under conditions that allow expression of the recombinant gene. In essence, they are standard culture conditions known to those of skill in the art, but in the case of cells containing the gene for human factor IX, vitamin K should be included in the medium.

The term “subject” as used herein refers to a human individual. This individual could be a patient requiring prophylaxis and/or medical treatment. In some embodiment, subject could be a mammal such as mouse, cow, rat, etc.

The term “vaccine” as used herein refers to a suspension of antigens derived from viruses or bacteria that, upon administration, will produce active immunity and provide protection against those viruses or bacteria or related viruses or bacteria.

The term “virus producing cell” as used herein refers to cell that a virus has infected and whose cell machinery the virus can direct to produce more viruses.

The term “virus or virion” as used herein refers to a submicroscopic infectious agent that is unable to grow or reproduce outside a host cell. It is non-cellular but consisting of a core of DNA or RNA surrounded by a protein coat. A virus is a small parasite that cannot reproduce by itself. Once it infects a susceptible cell, however, a virus can direct the cell machinery to produce more viruses. Virus and virion as used herein are synonymous.

The term “target cell” or “host cell” comprises any cell type, such as a mammalian cell, that is susceptible to transformation, transfection, or transduction, with a nucleic acid construct or vector. In some embodiments, the host cell, such as a mammalian cell, is a T cell or a T regulatory cell. In some embodiments, the host cell, such as a mammalian cell, is a hematopoietic stem cell. In some embodiments, the host cell is a CD34+ cell, e.g., a CD34+ hematopoietic stem cell. As used herein, the term “population of cells” refers to a group of cells, such as mammalian cells, comprising more than one cell. In some embodiments, a cell, such as a mammalian cell, is manufactured, wherein the cell comprises the protein sequence as described herein or an expression vector that encodes the protein sequence as described herein.

The term “T-cell” is a type of lymphocyte, which develops in the thymus gland and plays a central role in the immune response. T-cells can be distinguished from other lymphocytes by the presence of a T-cell receptor on the cell surface. “T cells” or “T lymphocytes” as used herein can be from any mammalian, e.g., primate, species, comprising monkeys, dogs, and humans. In some embodiments, the T cells are allogeneic (from the same species but different donor) as the recipient subject; In some embodiments the T cells are autologous (the donor and the recipient are the same); In some embodiments, the T cells are syngeneic (the donor and the recipients are different but are identical twins).

The term “Interferons or IFNs” are a group of signaling proteins made and released by host cells in response to the presence of several viruses. A virus-infected cell will release interferons causing nearby cells to heighten their anti-viral defenses.

The term “Interleukin (IL)”, any of a group of naturally occurring proteins that mediate communication between cells. Interleukins regulate cell growth, differentiation, and motility. They are particularly important in stimulating immune responses, such as inflammation.

The term “ligand” is a substance that forms a complex with a biomolecule to serve a biological purpose.

Term “virus of interest” could be a virus known to a person skilled in the art, more particularly it includes enveloped viruses. Enveloped viruses are a class of viruses that bud from the plasma or internal membrane of plants or animals during their replication. The newly budded viral particle contains the genomic material inside a protein capsid, which in turn is surrounded by membrane from the host and envelope proteins. Envelope proteins are often heavily glycosylated by the host machinery, and therefore are often not immediately recognized by the immune system. These envelope glycoproteins are usually involved in interactions with cellular receptors on target host cells, triggering membrane fusion and infection.

Enveloped viruses cause many well-known diseases, including influenza, Ebola, chicken pox, SARS (severe acute respiratory syndrome), small pox, and AIDS. (See, e.g., Fields, et al., cited above.) Human immunodeficiency virus (HIV), the virus that causes AIDS, affects approximately 33 million people throughout the world and causes approximately 2 million HIV-related deaths per year. (See, e.g., UNAIDS, Report On The Global AIDS Epidemic, 2008, the disclosure of which is incorporated herein by reference.) While current retroviral therapies have extended the length and quality of life of those infected with HIV, resistant strains are becoming increasingly common, and additional treatments and a broad spectrum vaccine are necessary to prevent additional infections. In turn, although influenza does not typically cause the mortality of HIV, it is a highly contagious virus that can be lethal, usually in the very young and very old, and in those with immune deficiencies. Moreover, influenza pandemics, such as the one in 1918 when an estimated 40 million people worldwide were killed, are capable of causing a significant number of deaths, including in healthy young adults. (See, e.g., Reid, A. H., et al., J Gen Virol, 84, 2285-92, 2003, the disclosure of which is incorporated herein by reference.)

Enveloped RNA viruses can encompass single-stranded RNA viruses (ssRNA) or double-stranded RNA viruses (dsRNA). Within the ssRNA viruses there are positive-sense (+ RNA) and negative-sense (−RNA) viruses. Representative families/members include bromoviruses (BMV), nidoviruses (SARS-CoV), picornaviruses (PV), calci viruses (MNV), alphaviruses (CHIKV), flaviviruses (DV), retroviruses (HIV-1), orthomyxoviruses (IVA), bunyaviruses (LCEV), arenaviruses (MACV), paramyxoviruses (MV), and rhabdoviruses (VSV).

In an embodiment, enveloped viruses are RNA viruses.

The term, “CD34+ cells” are immature cells expressing CD34 cell surface markers. CD34+ cells are considered to comprise a subpopulation of cells having the properties of stem cells.

In an embodiment, CD34+ cells could be collected from biological samples include, but are not limited to, peripheral blood, bone marrow and adipose tissue. In exemplary aspects, the biological sample is obtained from the donor via apheresis, e.g., but without limitation, leukapheresis. In exemplary aspects, the biological sample is the mononuclear fraction obtained from the donor via apheresis (e.g., but without limitation to, leukapheresis). In an embodiment, donor of CD34+ cell is a mammal. In specific aspects, the donor is a human. In some embodiments, the donor of the CD34+ is the same as the patient or the subject to be treated with the pharmaceutical compositions of the invention. In this regard, the CD34+ cell population is considered “autologous” to the patient or subject. In other embodiments, the donor of the cell population is different from the patient or subject to be treated, but the donor and patient are of the same species. In this regard, the cell population is considered as “allogeneic.”

The term “treatment,” when used in referring to a disease or condition, means that at least an amelioration of the symptoms associated with the condition afflicting an individual is achieved, where amelioration is used in a broad sense to refer to at least a reduction in the magnitude of a parameter, e.g., a symptom, associated with the condition (e.g., viral infection) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

Term “effective amount,” “pharmaceutically effective amount,” or “therapeutically effective amount” as used herein mean a sufficient amount of the composition to provide the desired utility when administered to a subject having a particular condition. In the context of ex vivo treatment of viral infection, the term “effective amount” refers to the amount of a population of virions or their progeny needed to prevent or alleviate at least one or more signs or symptoms of viral infection. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

A “personalized pharmaceutical” shall mean specifically tailored therapies for one individual patient that will only be used for therapy in such individual patient. In an embodiment, personalized pharmaceutical includes personalized vaccines and therapies using patients CD34 to produce viral particles.

CD34 is commonly used as a stem and progenitor cell marker. However, little is known about the exact function of CD34.

It has long been recognized that direct viral infection of CD34⁺ stem cells and progenitor cells is difficult and limiting (25-29). Some early studies failed to detect HIV-1 DNA in highly purified CD34⁺ cell population (30) or in Lin(−)/CD34(+) hematopoietic precursor cells in patients (31), while other studies have suggested that CD133⁺ hematopoietic progenitor cells harbor HIV genomes in a subset of patients on long-term ART (32). HIV is also found to directly infect CD34⁺ multipotent hematopoietic progenitor cells (HPCs), although viral replication is limited (25, 33).

We demonstrated that the ubiquitous stem cell marker CD34 is an antiviral protein limiting retroviral spreading infection and replication in CD34⁺ cells such as but not limited to stem and progenitor cells.

### Method

In an embodiment, we provided evidence that CD34 is an antiviral protein limiting viral spreading infection in CD34+ progenitor cells. Our studies demonstrated that surface CD34 can be incorporated into progeny virions released from infected CD34+ cells, and this incorporation inactivates virus infectivity through inhibition of particle attachment to target cells.

In an embodiment, present invention provides a method comprising co-expressing a viral vector of a virus of interest and a CD34 expression vector into a viral producing cell to produce virus particles such that CD34 incorporates into the virus particles; isolating the virus particles from the VPC; and blocking a viral infection; and wherein the method is configured for inhibiting the viral infection of the virus of interest.

In an embodiment, viral infection could be of an enveloped RNA virus, such as bromoviruses (BMV), nidoviruses (SARS-CoV), picornaviruses (PV), calci viruses (MNV), alphaviruses (CHIKV), flaviviruses (DV), retroviruses (HIV-1), orthomyxoviruses (IVA), bunyaviruses (LCEV), arenaviruses (MACV), paramyxoviruses (MV), and rhabdoviruses (VSV).

In an embodiment, viral infection is a retrovirus.

In an embodiment, CD 34 expression results in incorporation into the expressed virus.

In another embodiment, CD 34 expression results in incorporation into the virus already present in the infected cell.

In an embodiment, inhibitory method directed toward a method of inhibiting viral infection in a tissue culture setting with infected cells (e.g., HIV-1-infected lymphocytes).

In another embodiment, inhibitory method could be in an in vivo model. (e.g., HIV-infected macaques).

Yet in another embodiment, the method could be an ex vivo setting (e.g., removing HIV-1-infected cells from a subject and subjecting them to CD-34 treatment).

In an embodiment, the viral producing cell is a human cell line.

In an embodiment, the method could be in vitro method or ex vivo method.

In an embodiment, the expression vector is in an amount of about 0.5 ng to about 400 ng, such as 1 ng, 5 ng, 10 ng, 50 ng, 100 ng, 200 ng, 300 ng or more. The expression vector has appropriate promoters, enhancers, and other regulatory elements required for expression of CD34. Expression vector could be such as pCMV3 vector.

In an embodiment, the expression vector may have appropriate an indicator gene to assess transfection efficiencies. The indicator gene could be GFP or any other known to a person skilled in the art.

In an embodiment, the ratio of CD 34 expression vector and the viral vector is about 1:1 to 1:1000, such as 1:5, 1:10, 1:50, 1:100 or more.

In an embodiment, CD34 expression vector inhibits the viral infection in a dose dependent manner or time dependent manner. In some embodiment, CD34 expression vector inhibits the viral infection in dose dependent manner.

In an embodiment, method expresses sufficient CD34 in those directly infected with HIV-1 to a level that is capable of inhibiting HIV-1 infectivity. In an embodiment, amount of CD34 could be 0.5 ng/ml, 5 ng/ml, 10 ng/ml, 50 ng/ml, 100 ng/ml, 200 ng/ml, 400 ng/ml or more.

The viral particles are isolated from viral producing cells using methods that preserve the integrity thereof, such as not limited to gradient centrifugation, e.g., cesium chloride, sucrose and iodixanol, as well as standard purification techniques including, e.g., ion exchange and gel filtration chromatography as known to a person skilled in the art.

In one embodiment, the invention comprises purified viral particles of the invention. In another embodiment, said viral particles isolated from VPC are at least 50%, 55% 60%, 65%, 70%, 75%, 80%, 85%, 90%, 95%, 96%, 97%, 98%, 99% or greater, free from other molecules (exclusive of solvent) in a mixture. In another embodiment, said viral particles of the invention are substantially free of other viruses, proteins, lipids, and carbohydrates associated with making viral particles of the invention.

In an embodiment, wherein the virus comprising the overexpressed CD34 protein comprises more than one copy, two copies, 5 copies, 10 copies, 20 copies, 50 copies, 70 copies, 100 of CD34 protein.

In an embodiment, overexpression encompasses about 1.5×, 2×, 3×, 5×, 10×, 50× or 100× more copies of CD34 than would normally be expressed by the cell on its own.

In an embodiment, a viral infection of the virus of interest is blocked using viral particles isolated from VPC. In another embodiment, said viral particles isolated from VPC could block the viral infection at least 50%, 55% 60%, 65%, 70%, 75%, 80%, 85%, 90%, 95%, 96%, 97%, 98%, 99% or greater.

The term, “blocking” or “inhibiting” the viral infection are interchangeably used.

In an embodiment, CD34+ hematopoietic stem cells and progenitor cells can serve as a distinct HIV reservoir that contributes to HIV persistence in ART-suppressed patients (34, 35).

In an embodiment, detailed mechanistic studies of the role of CD34 in cell line model systems demonstrates that: (1) CD34 does not intrinsically block viral entry and release, allowing viral entry, integration, and gene expression to occur in CD34+ cells; (2) a dominant phenotype of CD34 is the inactivation of progeny virions released, limiting spreading viral replication in CD34+ cell population; (3) our study also suggests a unique potential of HIV+CD34+ cells for producing infectious virus following cell differentiation and loss of surface CD34 (34, 36, 37).

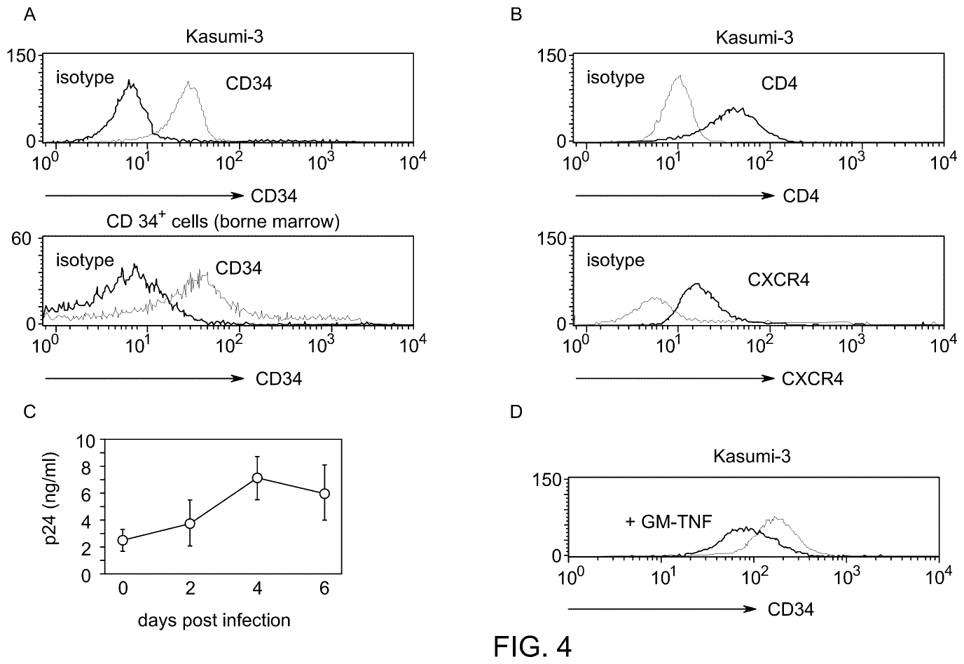

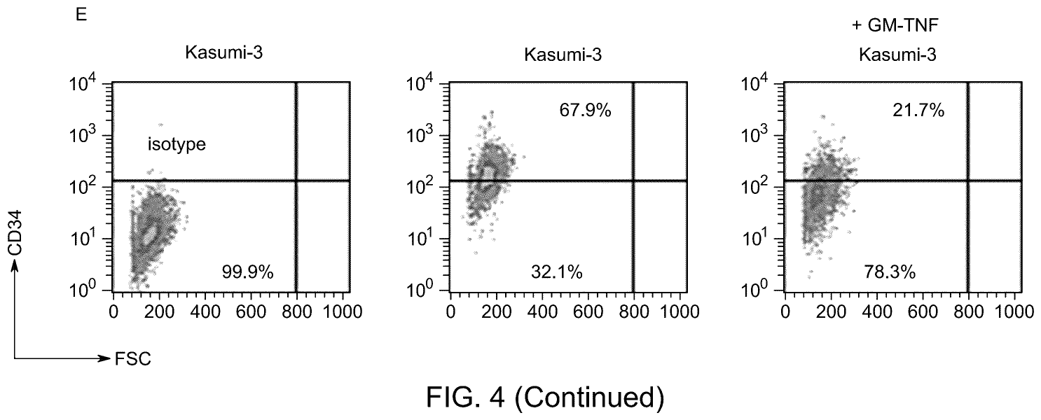

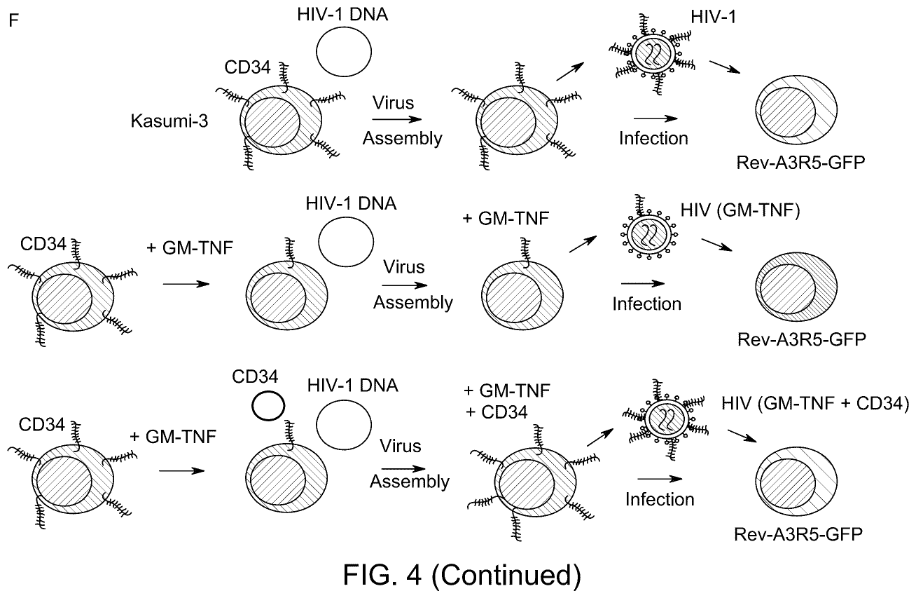

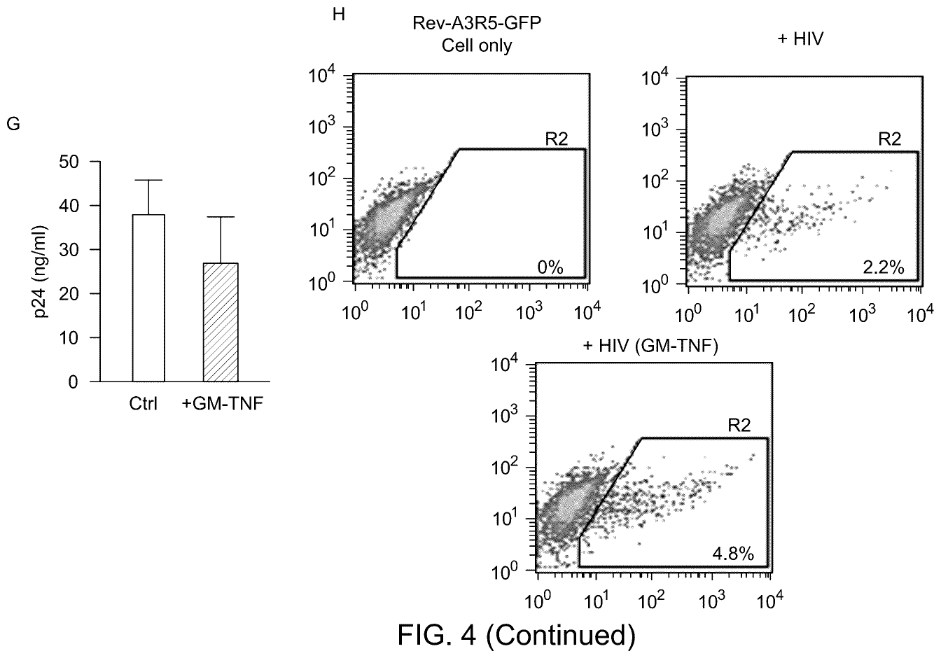

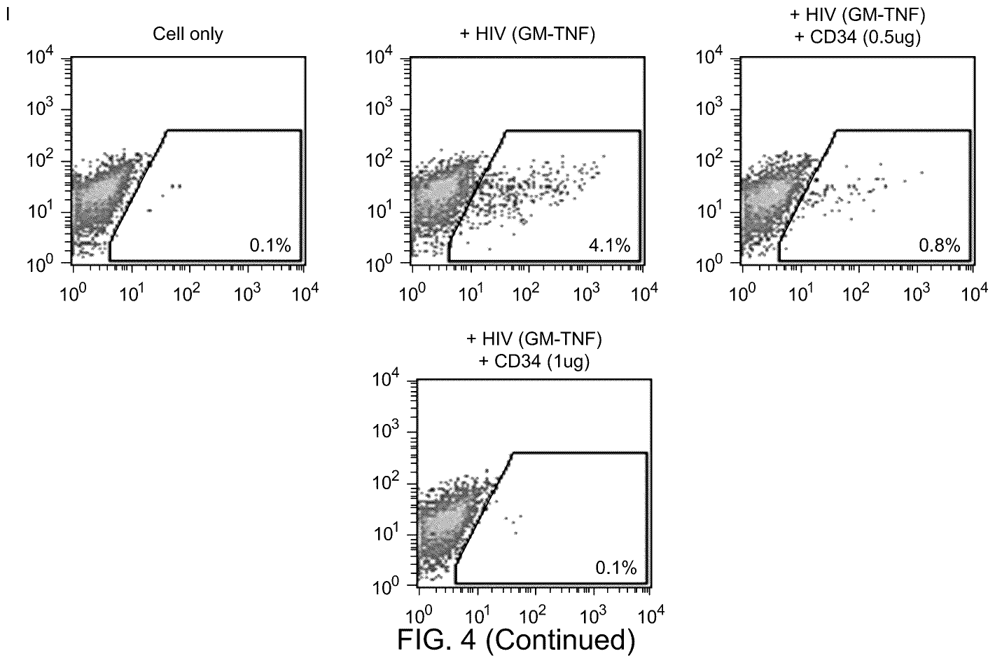

In an embodiment, studies are conducted mainly in CD4 T cells and CD34+ Kasumi-3 cell. We recognize that currently there are limited CD34+ cell lines suitable as model systems to study HIV infection of CD34+ cells. Our initial screening of CD34+ cell lines confirmed that Kasumi-3 naturally expresses CD34 and the HIV receptor and co-receptor, CD4 and CXCR4 (FIGS. 4A and 4B). Kasumi-3 has also been reported to carry many of the characteristics of undifferentiated leukocytes, and was considered a progenitor cell that can respond to the induction by stem cell factors (24). Importantly, our study also discovered that Kasumi-3 similarly responded to GM-CSF/TNF-alpha induction which leads to CD34 downregulation (FIGS. 4D and 4E), a characteristic shared with primary hematopoietic progenitor cells (25).

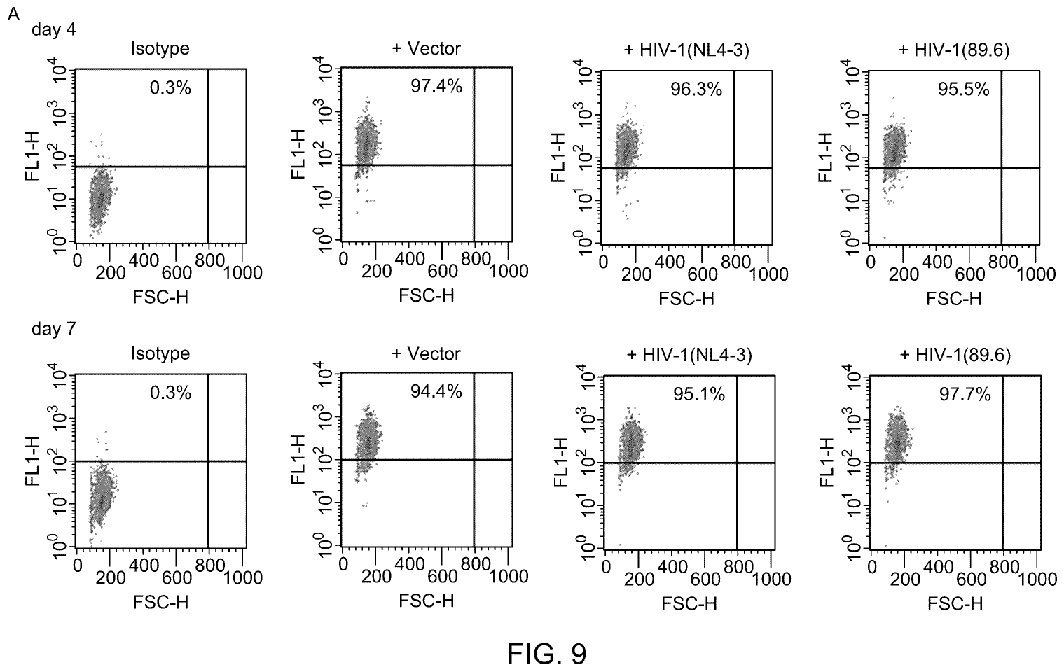

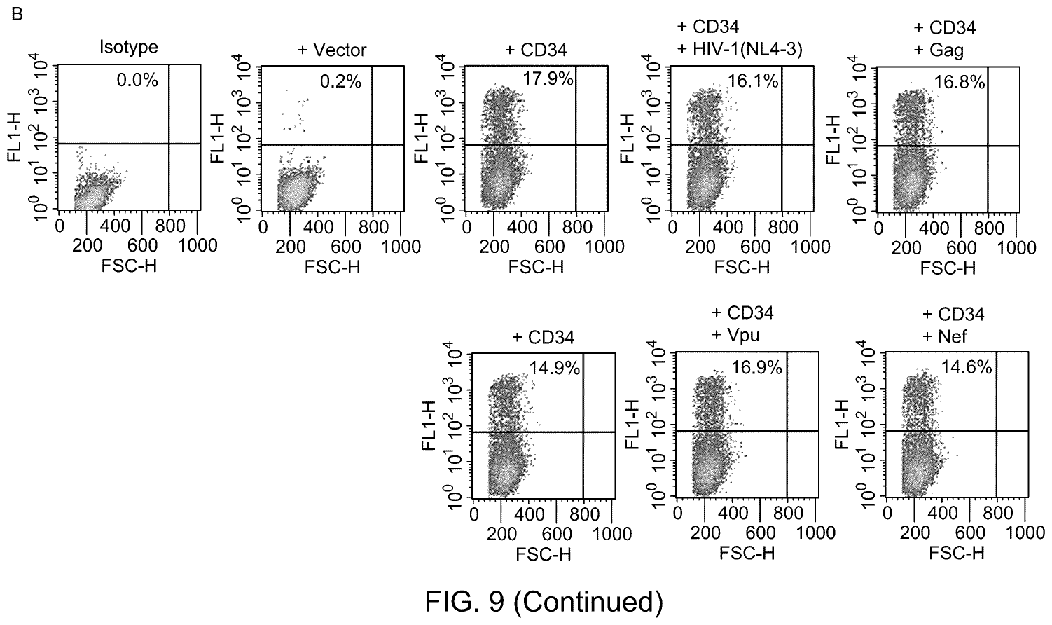

Interestingly, although HIV can use accessory proteins such as Vpu and Nef to antagonize mucin-like proteins such as PSGL-1 (13, 14), the virus is not capable of downregulating CD34 (FIG. 9), suggesting an intrinsic nature of stem and progenitor cells to limit retroviral dissemination in this critical cell population. It is possible that the ubiquitous presence of CD34 is a part of the innate immunity protecting stem and progenitor cells from some enveloped viruses such as certain retroviruses.

In an embodiment, the invention provides an ex vivo method comprising: taking CD34+ cells; transfecting a viral vector expressing a virus of interest into the CD34+ cells; producing viral particles in the CD34+ cells and isolating the virus particles from the CD34+ cells, and wherein the viral particle are non-infectious virus particles, attenuated virus particles or inactivated virus particles or combination thereof of the virus of interest.

### Composition

In an embodiment, present invention provides composition comprising a recombinant virus comprising an overexpressed CD34 protein; wherein the virus particle comprises a non-infectious virus particle, an attenuated virus particle or an inactivated virus particle.

The composition is a pharmaceutical composition. The pharmaceutical compositions useful herein contain a pharmaceutically acceptable carrier, including any suitable diluent or excipient, which includes any pharmaceutical agent that does not itself induce the production of an immune response harmful to the animal receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in animals, and more particularly in humans. These compositions can be useful as a vaccine and/or immunogenic compositions for inducing a protective immune response in an animal.

In an embodiment, the composition can contain excipients, such as buffers, binding agents, blasting agents, diluents, flavors, lubricants, etc. The peptides can also be administered together with immune stimulating substances, such as cytokines.

An extensive listing of excipients that can be used in such a composition can be, for example, taken from A. Kibbe, Handbook of Pharmaceutical Excipients (Kibbe, 2000).

In an embodiment, composition is used for parenteral administration, such as subcutaneous, intradermal, intramuscular. In an embodiment, composition is used for oral administration such as tablets, capsules etc. In another embodiment, composition may be used as nasal spray etc.

The present invention also relates to a medicament comprising a virus particle as defined above as an active ingredient. The virus particle is a recombinant virus particle. The recombinant virus particle comprising the overexpressed CD34 protein comprises more than one copy of CD34 protein, such as it may contain 2 copies, 4 copies, 8 copies, 10 copies, 20 copies, 100 copies, or more copies of CD34 protein.

In an embodiment, recombinant virus particles are non-infectious virus particles, attenuated virus particles or inactivated virus particles or combination thereof.

In an embodiment, the present study suggests that the ubiquitous presence of CD34 on stem and progenitor cells is likely a part of innate immunity against some enveloped viruses such as retroviruses.

In an embodiment, the composition may be used as a vaccine. It is important to realize that the immune response triggered by the vaccine according to the invention attacks a disease such as viral in different cell-stages and different stages of development.

### Materials and Methods

### Cells and Cell Culture.

HEK293T (ATCC) and NIH/3T3 (ATCC) cells were maintained in Dulbecco's modified Eagle's medium (DMEM) (Thermo Fisher Scientific). HIV Rev-dependent GFP indicator Rev-A3R5-GFP cells (kindly provided by Virongy Biosciences) were cultured in RPMI-1640 plus 10% FBS. Kasumi-3 cells (ATCC) were maintained in RPMI-1640 plus 30% FBS. HeLa (ATCC) and HeLa JC.53 cells (kindly provided by Dr. David Kabat) were maintained in Dulbecco-modified Eagle's medium (DMEM) (Thermo Fisher Scientific) containing 10% FBS and 1× penicillin-streptomycin (Thermo Fisher Scientific). Primary human bone marrow CD34+ cells were purchased from Stemcell Technologies.

### Plasmid Transfection and Virus Production.

HIV-1 DNA vectors, pHIV-1(NL4-3) and pHIV-1(89.6), were obtained from the NIH AIDS Reagent Program. The env-defective pNL4-3 derivative pNL4-3/KFS and the HIV-1 Env expression vector, pNLΔΨEnv, were described previously (21, 23). pCMV3-CD34 and the pCMV3-Empty vector were purchased from Sino Biological. The MLV packaging plasmid, pCL-Eco, was obtained from Addgene. The GFP-expressing retroviral vector pRetroQAcGFP1-N1 was purchased from Clontech. For HIV-1 virus production, HEK293T cells were cotransfected in 6-well plates with 1 μg of pHIV-1(NL4-3) plus the indicated doses of pCMV3-CD34 or pCMV3-Empty. Supernatants were harvested at 48 hours post cotransfection. HIV viruses were also assembled 10 cm dishes by transfection of HEK293T cells with 10 μg of pHIV-1(NL4-3) or pHIV(89.6). Supernatant was collected at 48 hours post transfection. For MLV-GFP virus production, HEK293T cells were cotransfected in 6 well plates with 1 μg of pCL-Eco and 1 μg of pRetroQAcGFP1-N1 plus the indicated doses of pCMV3-CD34 or the pCMV3-Empty vector. Supernatants were collected at 48 hours post cotransfection. For HIV(KFS)(Env) virus production, HEK293T cells were transfected in 10-cm dishes with 5 μg of pNL4-3/KFS and 5 μg of pNLΔΨEnv, and the supernatant was harvested at 48 hours post transfection. For HIV-1 p24 release assays, cells were cotransfected with 1 μg of HIV-1(NL4-3) DNA and the indicated amounts of pCMV3-CD34 or pCMV3-Empty vector using Lipofectamine 2000 (Invitrogen). Supernatant was collected at 48 hours post transfection.

### Virus Infectivity Assays.

For quantifying HIV-1 infectivity, HIV-1 particles were produced in the presence of pCMV3-CD34 or pCMV3-Empty. Particles were harvested at 48 hours, normalized by p24 content, and used to infect Rev-A3R5-GFP cells (0.2 to 0.5 million cells per infection). The percentage of GFP+ cells was quantified by flow cytometry at 48 to 72 hours post infection. For the MLV-GFP virus infectivity assay, NIH/3T3 cells were pretreated with Infectin II (provided by Virongy Biosciences) for 1 hour as suggested by the manufacturer. Cells were infected with MLV-GFP virus in the presence of Infectin II. The percentage of GFP+ cells was quantified by flow cytometry at 72 hours post-infection. To determine the inhibition of HIV-1 replication in CD34+ human CD4 T cells, Rev-A3R5-GFP cells (1 million cells) were electroporated with 500 ng of pCMV3-CD34 or pCMV3-empty using a Cell Line Nucleofector Kit R (Lonza). After 2 days post-electroporation, Rev-A3R5-GFP cells were infected with p24-normalized HIV-1(NL4-3) or HIV(KFS)(Env) virus. The percentage of GFP+ cells was quantified by flow cytometry at 48 to 72 hours post infection. HIV-1(89.6) virus was used to infect Kasumi-3 cells. Briefly, cells were pretreated for 1 hour with Infectin (Virongy). Virus particles were used to infect Kasumi-3 cells in the presence of Infectin. After 4 hours, infected cells were washed and cultured in medium. Infection supernatants were harvested at days 0, 3, 5, 7, 10 post infection for p24 ELISA.

To determine the infectivity of HIV-1 virus particles produced from GM-CSF plus TNF-α-cultured Kasumi-3 cells, cells were cultured with or without recombinant human GM-CSF (R&D systems) (100 ng/ml) plus recombinant human TNF-α (Biolegend) (2.5 ng/ml) for 7 days. Cells (2 million) were then electroporated with HIV-1 DNA (4 μg) or HIV-1 DNA plus a CD34 expression vector (0.5 and 1 μg) using a Cell Line Nucleofector Kit R (Lonza). Following electroporation, cells were cultured either in medium or with GM-CSF plus TNF-α for 4 days. Virus supernatant was harvested and used to infect Rev-A3R5-GFP indicator cells to measure virus infectivity. The percentage of GFP+ cells was quantified by flow cytometry (FACSCalibur, BD Biosciences).

### CD34 Virion Incorporation Assay.

To determine CD34 virion incorporation, HIV-1 particles assembled in the presence of pCMV3-CD34 or pCMV3-empty at the indicated dosage were harvested at 48 hours post-transfection in HEK293T cells. As a control, cells were also transfected with pCMV3-CD34 only. Transfection supernatant were filtered through 0.45 μm filter and purified by ultra-speed centrifugation through a 6%-18% OptiPrep gradient (13). Briefly, particle- or virus-containing supernatant was first concentrated (4° C., 7,500×g for 30 min) using Sartorius Vivaspin20 concentrator (Fisher Scientific). Concentrated viruses were purified by ultra-speed centrifugation through a gradient of 6-18% OptiPrep (Sigma-Aldrich) solution using 40,000 rpm at 4° C. for 2 hours (SW41Ti rotor from Beckman). Eleven individual fractions (1 ml per fraction) were collected, and then pelleted by a second round of ultracentrifugation (20,000 rpm, 4° C. for 1.5 hours, SW41Ti). Pellet from each fraction was resuspended in 100 μl LDS lysis buffer (Thermo Fisher Scientific) for western blot detection of proteins. CD34 and viral p24 proteins in each fraction were analyzed by western blot using antibodies against CD34 (Clone 563, BD Pharmingen) or HIV-1 p24 (mouse anti-HIV-1 p24 monoclonal antibody, clone 183-H12-5C, NIH AIDS Reagent Program). For the magnetic beads pull-down assay, HIV-1 particles were assembled in 6-well plates by cotransfection of HEK293T cells with pHIV-1(NL4-3) DNA (1 μg) plus pCMV3-CD34 or pCMV3-empty at the indicated dosage. Particles were harvested at 48 hours post-transfection, normalized for HIV-1 p24, and subjected to immuno-magnetic capture as previously described (18). Briefly, magnetic Dynabeads Pan Mouse IgG (Invitrogen) were conjugated with anti-human CD34 antibody (Clone 563, BD Pharmingen) or purified Mouse IgG1, K Isotype control antibody (clone MG1-45, Biolegend) for 30 min at room temperature. After conjugation, the antibody-conjugated beads were incubated with p24 normalized CD34-incorporated viral particles for 1 hour at 37° C. The complex was pulled down with a magnet and washed with cold PBS twice. Captured viral particles were eluted in 10% Triton x-100 PBS, diluted, and quantified by p24 ELISA. To determine whether CD34 incorporation affects the incorporation of the HIV envelope, HIV-1 particles produced in the presence of pCMV3-CD34 or pCMV3-empty at the indicated dosage were harvested 48 hours post-transfection in HEK293T cells. Particles were purified through 10% sucrose gradient centrifugation. Particles were analyzed by western blot using antibodies against HIV gp41 (human anti-HIV-1 gp41 antibody, clone 2F5, NIH AIDS Reagent Program) and HIV p24 (mouse anti-HIV-1 p24 monoclonal antibody, 183-H12-5C, NIH AIDS Reagent Program).

### Viral Attachment Assay.

HIV virion particles produced in the presence of pCMV3-CD34 or pCMV3-empty were incubated with Rev-A3R5-GFP cells, HeLa cells, or HeLa JC.53 cells (prechilled at 4° C. for 1 hour) at 4° C. for 2 hours. The cells were then washed extensively (5 times) with cold PBS buffer and then lysed with LDS lysis buffer (Thermo Fisher Scientific) for analysis by western blot.

### Viral Entry Assay.

HIV-1 entry assay (beta-lactamase-based viral entry assay or BlaM) was performed as previously described (38). Briefly, viruses were generated by cotransfection of HEK293T cells with three plasmids: pHIV-1(NL4-3), pAdvantage (Promega) and pCMV4-3BlaM-Vpr (kindly provided by Dr. Warner C. Greene) at a ratio of 6:1:2. Supernatant was harvested at 48 hours post-transfection, concentrated, and then used for infection of Rev-A3R5-GFP cells that have been electroporated with pCMV3-CD34 or pCMV3-empty. β-lactamase, and CCF2 (LiveBLAzer™ FRET-B/G Loading Kit with CCF2-AM, Invitrogen) measurements were performed using a 407-nm violet laser with emission filters of 525/50 nm (green fluorescence) and 440/40 nm (blue fluorescence), respectively.

### Surface Staining.

For CD34 surface staining, cells were stained with mouse anti-human CD34 (Clone 563, BD Pharmingen) followed by staining with PE-cyanine5-labeled F(ab′)2-goat anti-mouse IgG (H+L) secondary antibody (Invitrogen). For CD4, CXCR4, CD34 surface staining, cells were stained with mouse anti-human CD34 (Clone 563, BD Pharmingen), mouse anti-human CD184 (Clone 12G5, BD Pharmingen), or mouse anti-human CD4 (Clone RPA-T4, BD Pharmingen) followed by staining with AF488-labeled goat anti-mouse antibody (Invitrogen). Cells were also stained with mouse anti-human CD162 (BD Pharmingen, clone KPL-1), mouse anti-human CD43 (BD Pharmingen, clone 1G10), mouse anti-human CD34, (BD Pharmingen, clone 563), human podocalyxin antibody (R&D Systems, clone 222328), human endoglycan/PODXL2 antibody (R&D Systems, clone 211805), TMEM123 monoclonal antibody (Invitrogen, clone 297617), anti-human CD164 antibody (Biolegend, clone 67D2), human TIM-1/KIM-1/HAVCR antibody (R&D Systems, clone #219211), mouse anti-human MUC1 (CD227) (BD Pharmingen, Clone HMPV), or human MUC-4 antibody, (R&D Systems, Clone 781631). Following primary antibody staining, cells were also stained with Alexa Fluor 488 goat anti-mouse IgG secondary antibody (2 mg/ml, Invitrogen) (1:10 dilution).

### EXAMPLES

### Example 1: Virion Incorporation of CD34 Inactivating HIV-1 Infectivity

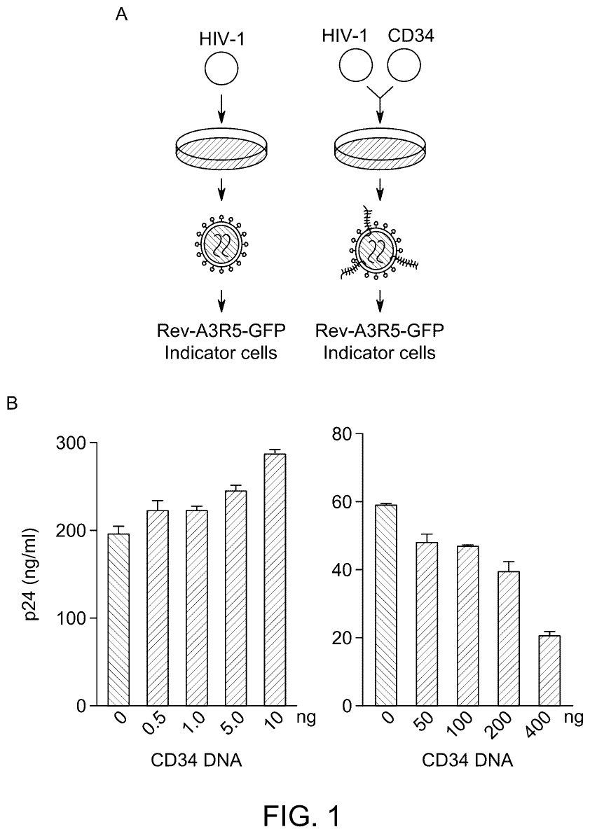

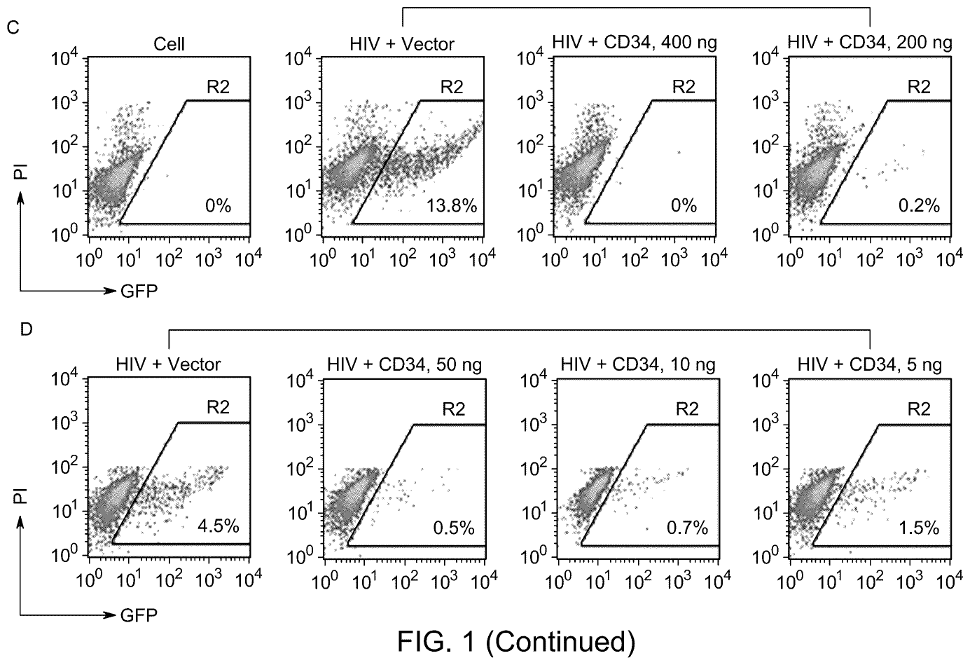

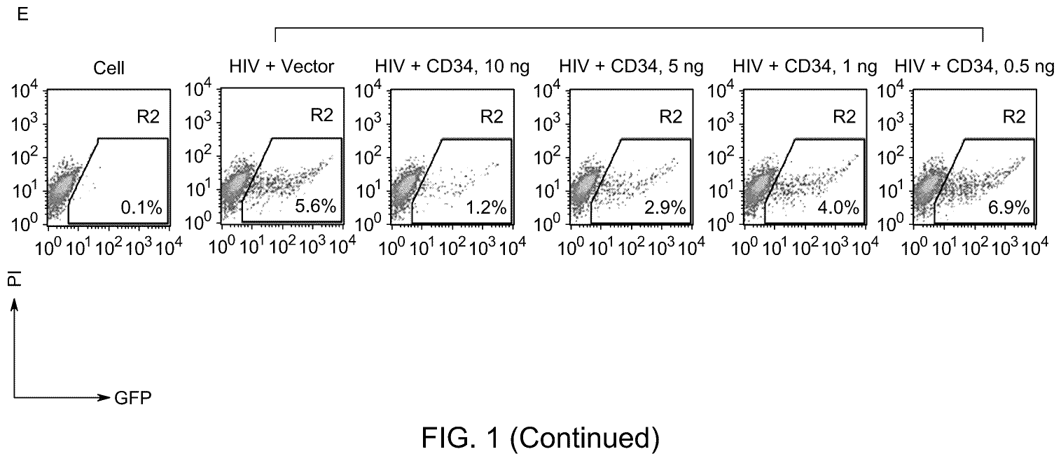

To investigate a possible role of CD34 in diminishing HIV virion infectivity, we first assembled HIV-1 particles in the presence of CD34; HEK293T producer cells were cotransfected with a CD34-expressing vector and HIV-1 DNA to produce particles (FIG. 1A). We tested a range of CD34 DNA dosages, from 0.5 ng to 400 ng, and found that at low dosages (0.5-10 ng), CD34 did not inhibit virus release from producer cells. At higher dosages, CD34 only slightly inhibited virion release (approximately 50% inhibition at 400 ng) (FIG. 1B). We further quantified the infectivity of progeny virions released using the HIV Rev-dependent GFP indicator cell Rev-A3R5-GFP (13, 16, 17). When an equal p24 level of input virus was used, we observed near-complete inhibition of HIV virion infectivity at CD34 dosages of 10 ng and above. CD34 partially inhibited HIV-1 infectivity at inputs as low as 1 ng (CD34-to-HIV DNA ratio, 1:1000), and there was a dose-dependent inactivation of HIV-1 at CD34 vector doses from 1 to 10 ng. The IC₅₀ was estimated to be at around 2-5 ng of CD34 (FIG. 1C-1E) (18).

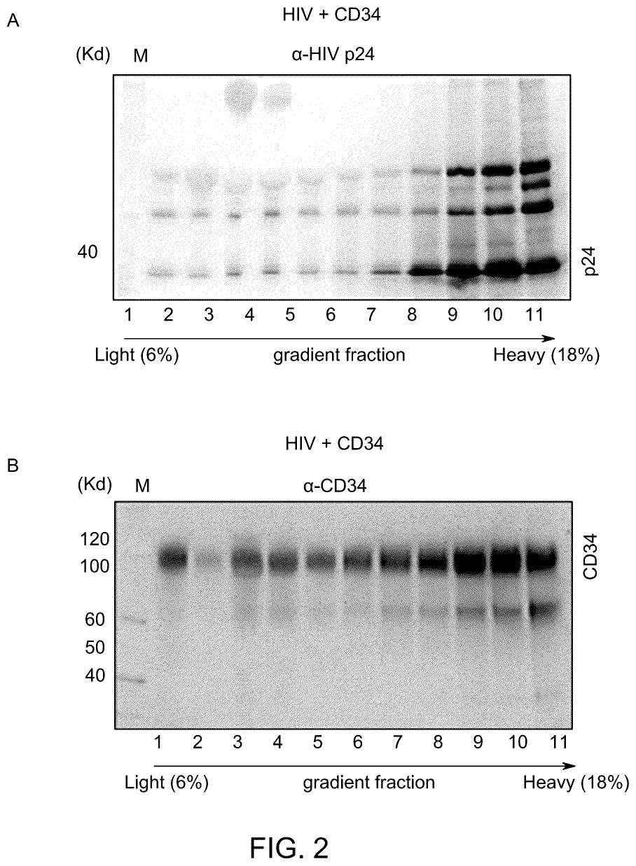

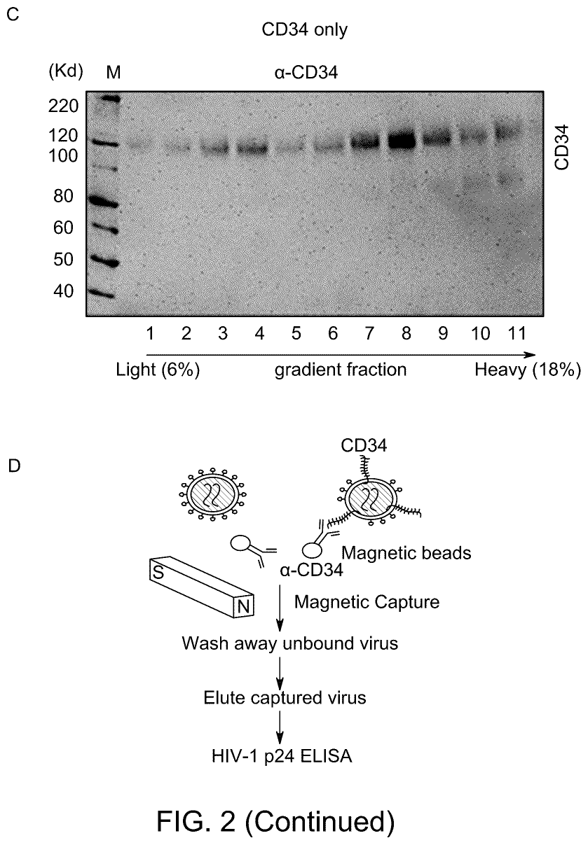

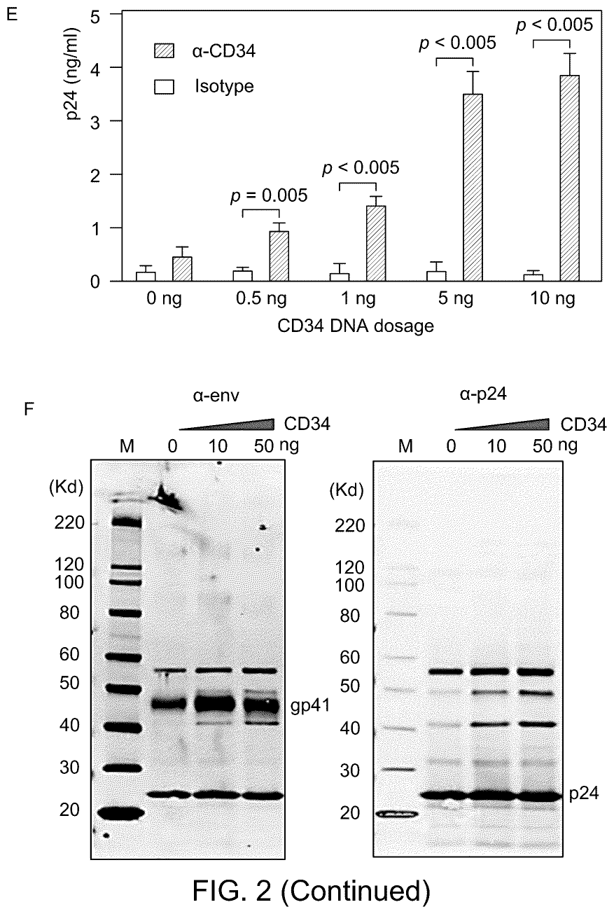

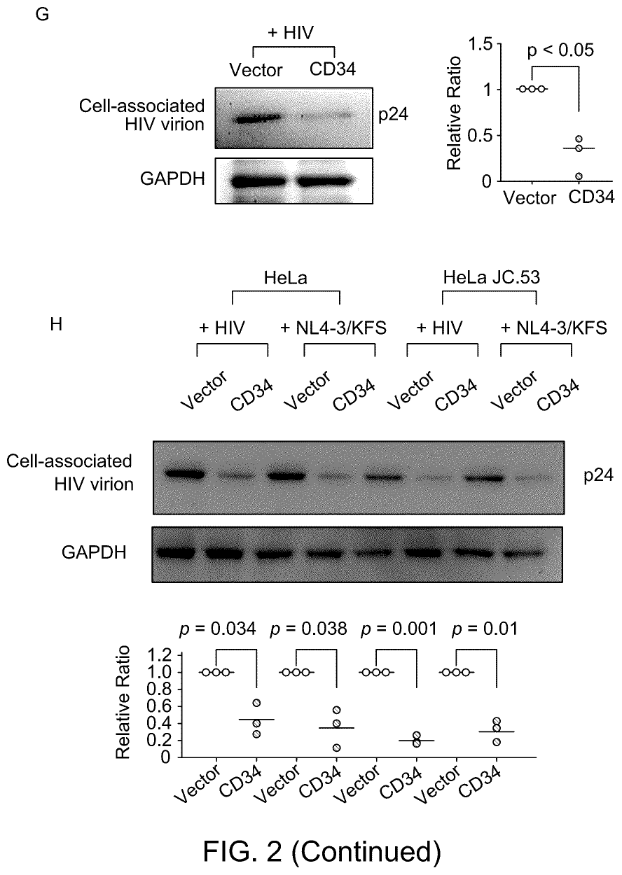

Mechanistically, the inhibition of virion infectivity likely results from virion incorporation of CD34 that may sterically hinder virus particle attachment to target cells and/or block HIV Env virion incorporation, mechanisms that may be similar to PSGL-1-mediated inactivation of virion infectivity (13, 15). To detect virion incorporation of CD34, we performed density gradient centrifugation to purify virions released from cotransfected HEK293T cells and observed co-sedimentation of CD34 with purified virions only when cells were cotransfected with the CD34 vector plus HIV DNA (FIGS. 2A and 2B), but not with the CD34 vector alone (FIG. 2C).

We further confirmed CD34 virion incorporation using a particle pull-down assay (18); anti-CD34 antibody-conjugated magnetic beads were used to pull down virion particles that express CD34 on the surface (FIG. 2D). Magnetically-purified virions were further quantified for the presence of HIV-1 p24 in the virions. We were able to demonstrate that only the anti-CD34 antibody, but not the control non-specific antibody, selectively pulled down p24⁺ virion particles in a CD34-dosage dependent manner, confirming virion incorporation of CD34 (FIG. 2E).

We also investigated the effects of CD34 expression in virus-producer cells on virion Env incorporation. We did not observe that CD34 inhibited HIV-1 Env incorporation at the dosages tested (FIG. 2F). We further performed a virion attachment assay and observed that CD34-imprinted virions were inhibited in their ability to attach to Rev-A3R5-GFP target cells (FIG. 2G). To determine whether this inhibition of virion attachment is dependent on specific interaction of HIV Env with the CD4 receptor and co-receptor, we performed virion attachment assay using CD4-positive and CD4-negative cells (HeLa JC.53 and HeLa), and observed that the presence of CD34 inhibited virion attachment to both (FIG. 2H), demonstrating that the inhibition of virion attachment is not dependent on interaction of Env with the CD4 receptor. In addition, we took advantage of previous findings that HIV particles can bind target cells even in the absence of Env-receptor interaction (19, 20). We assembled HIV particles devoid of any viral envelope glycoproteins (NL4-3/KFS) (21) in the presence or absence of CD34, and performed virion attachment assays. As shown in FIG. 2H, we observed that CD34-imprinted, Env-negative particles were also impaired in their ability to attach to HeLa and HaLa JC.53 cells. These results confirmed that CD34 inhibits virion attachment to cells in an envelope glycoprotein-independent manner. Thus, we speculate that the anti-retrovirus activity of CD34 is likely pleotropic.

### Example 2: Expression of CD34 in Human CD4 T Cells Elicits Anti-HIV-1 Activity

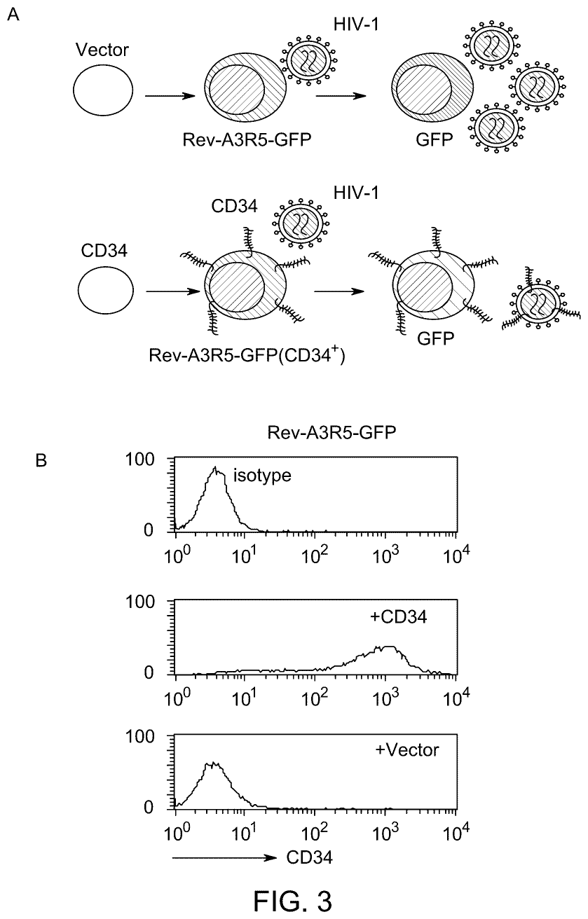

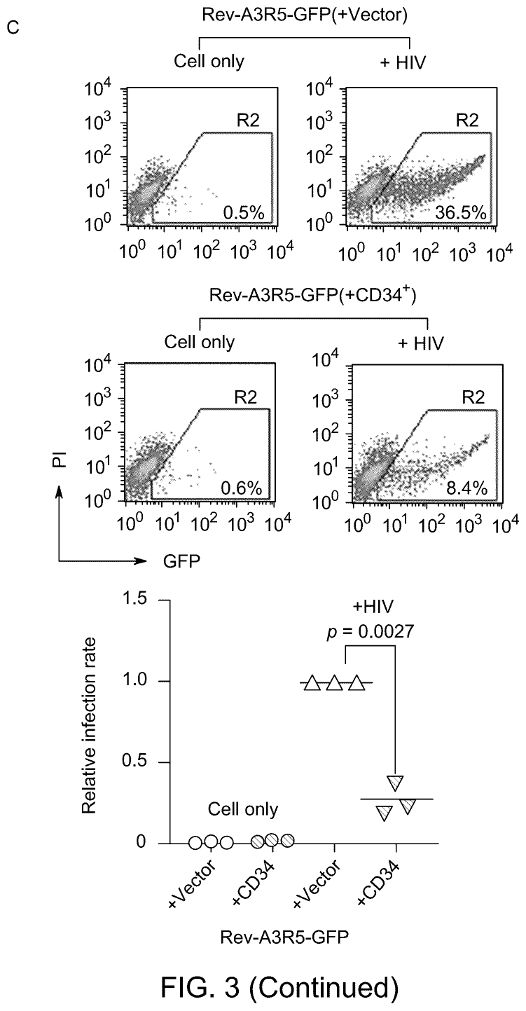

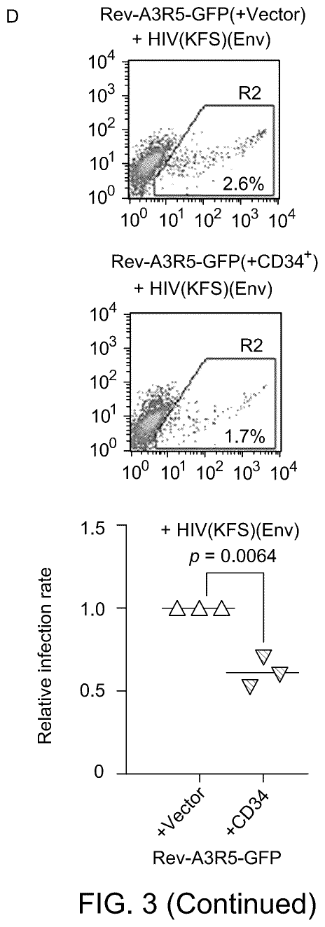

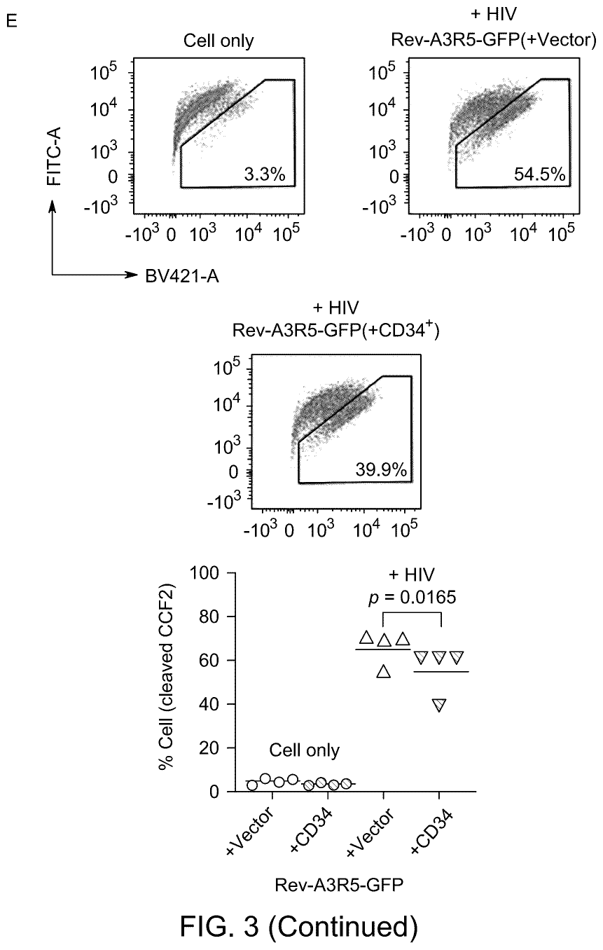

To confirm that CD34 has antiviral activity in HIV-1 target CD4 T cells, we transiently expressed CD34 in a human CD4⁺ lymphoblastoid cell line, Rev-A3R5-GFP (16, 22) (FIG. 3A). Following CD34 vector electroporation, surface expression of CD34 on Rev-A3R5-GFP was confirmed by surface staining (FIG. 3B). Cells were subsequently infected with HIV-1, and viral replication was monitored by HIV-1-mediated GFP expression. We observed that expression of CD34 inhibited HIV-1 replication in CD34⁺CD4⁺ T cells, reducing the percentage of HIV-infected GFP⁺ CD4 T cells from 35.6% to 8.4% (a 76.4% reduction) (FIG. 3C). The experiments were repeated 3 times, and on average, there was a statistically significant 72.6% reduction of HIV infection of CD34⁺CD4⁺ T cells.

We also followed a single HIV-1 infection cycle, and infected the CD34⁺CD4⁺ T cells with an HIV-1 Env-pseudotyped, single-cycle virus, HIV(KFS)(Env) (21, 23). Expression of CD34 reduced viral infection to approximately 65% of the empty vector control (a 34.6% reduction) (FIG. 3D). We also repeated the experiment 3 times, and on average, there was a statistically significant 38.9% reduction in HIV(KFS)(Env) infection of CD34⁺CD4⁺ T cells. This slight reduction of single-round infection likely resulted from inhibition of viral entry. We performed beta-lactamase-based viral entry assays (4 independent assays) and found that on average, expression of CD34 on the surface of the CD4 T cells reduced viral entry from 64.9% to 55% (a 15% reduction). Nevertheless, this slight reduction in viral entry may not be statistically significant (p=0.165) (FIG. 3E). Given that virion incorporation of CD34 blocks particle attachment to target cells (FIGS. 2G and 2H), and cell surface expression of CD34 minimally blocks viral entry (FIG. 3E), it is likely that CD34 inhibits spreading viral replication mainly through inactivating progeny virions released from CD34+ cells.

### Example 3: Physiological Levels of CD34 Limit HIV-1 Replication in CD34+ Hematopoietic Progenitor Kasumi-3 Cell

To confirm that CD34 naturally expressed on stem and progenitor cells possesses antiviral activity, we examined an undifferentiated leukemia progenitor cell, Kasumi-3 (24), which naturally expresses CD34 and the HIV receptor and co-receptor, CD4 and CXCR4 (FIGS. 4A and 4B). When levels of CD34 on Kasumi-3 were compared with those on primary human bone marrow CD34⁺ cells, we observed comparable peak levels of CD34 (FIG. 4A). In addition, the whole population of kasumi-3 is CD34⁺, whereas CD34 expression on bone marrow CD34⁺ cells is more heterogeneous (FIG. 4A).

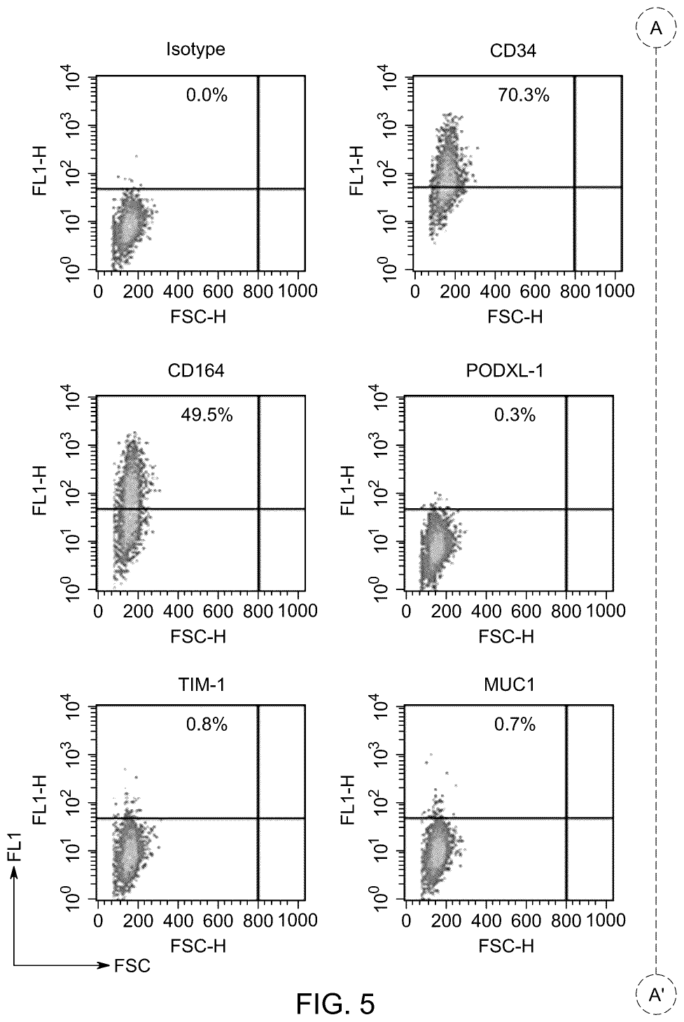

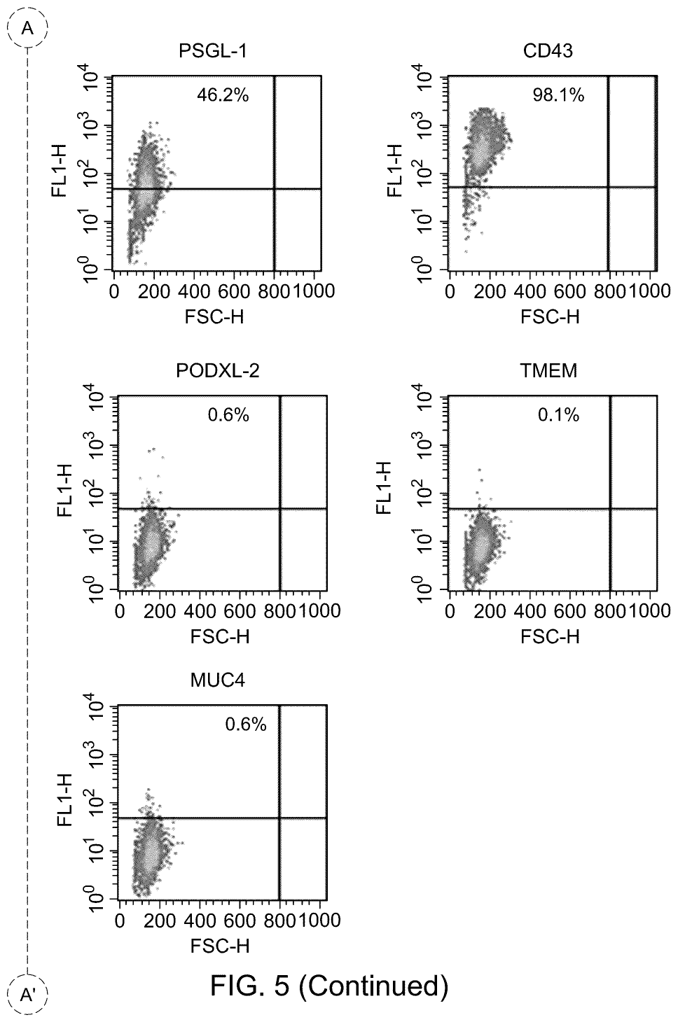

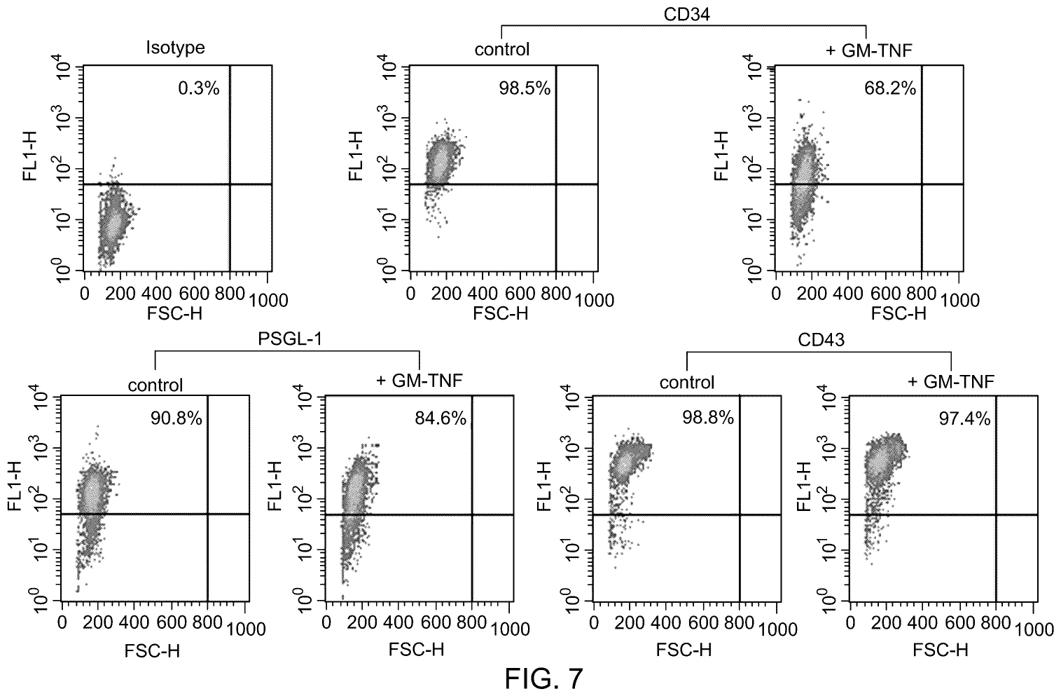

We found that in addition to CD34, Kasumi-3 also expresses multiple mucin-like surface glycoproteins such as PSGL-1, CD43, and CD164 that have all been shown to possess antiviral activities (13-15, 18) (FIG. 5). We infected or transfected Kasumi-3 cells with HIV-1 virus or viral DNA, both HIV-1(NL4-3) and HIV-1(89.6), and detected low-levels of viral release; however, there was no spreading viral replication (FIG. 4C and FIG. 6), in agreement with a previous finding showing that HIV-1 can enter and express genes in CD34⁺ multipotent hematopoietic progenitor cells (HPCs), but viral replication is limited (25). To investigate the role of CD34 in HIV-1 infection of Kasumi-3 cells, we took advantage of a previous finding that culturing hematopoietic progenitor cells (HPCs) in GM-CSF and TNF-α can induce myeloid differentiation and downregulation of CD34 from the cell surface (25). We cultured Kasumi-3 in GM-CSF and TNF-□, and demonstrated that GM-CSF/INF-□ induced downregulation of CD34 but not PSGL-1 or CD43 (FIGS. 4D and 4E, and FIG. 7).

To determine the effects of GM-CSF/TNF-□-induced CD34 downregulation on the infectivity of progeny virions, we cultured Kasumi-3 in GM-CSF and TNF-□ for 7 days, and then electroporated HIV-1 DNA into cells to assemble virion particles (FIG. 4F); we used electroporation rather than infection to eliminate possible additional effects of CD34 on viral entry. As a control, viral particles were also assembled in the absence of GM-CSF and TNF-□. Culturing Kasumi-3 cells in GM-CSF and TNF-□ did not enhance viral release (FIG. 4G). However, when the infectivity of the virions released was quantified on the Rev-A3R5-GFP reporter cells, using an equal p24 level of input virus, we observed enhanced infectivity of virions released from GM-CSF/TNF-□-cultured Kasumi-3 cells (FIG. 4H). To determine whether downregulation of CD34 by GM-CSF and TNF-□ may contribute to GM-CSF/TNF-□-mediated enhancement of virion infectivity, we reintroduced CD34 in GM-CSF/TNF-□-cultured Kasumi-3 cells using a CD34 expression vector with constitutive CMV promoter (FIG. 4H). Expression of CD34 in GM-CSF/TNF-□-cultured Kasumi-3 cells diminished virus infectivity enhanced by GM-CSF/TNF-□ (FIG. 4I). It is possible that multiple factors may be involved in the enhancement of virion infectivity by GM-CSF/TNF-□. Nevertheless, our results suggested that CD34 could be one of the factors related to GM-CSF/TNF-□-mediated enhancement of virion infectivity. Collectively, our results suggest that expression of CD34 in Kasumi-3 can limit HIV replication.

### Example 4: Broad Spectrum Antiviral Activity

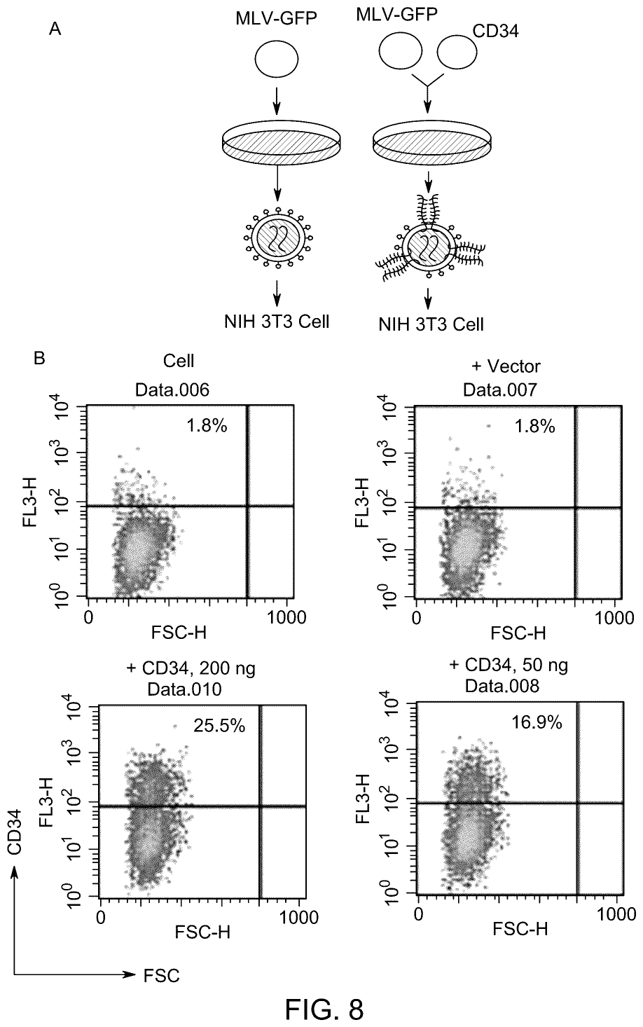

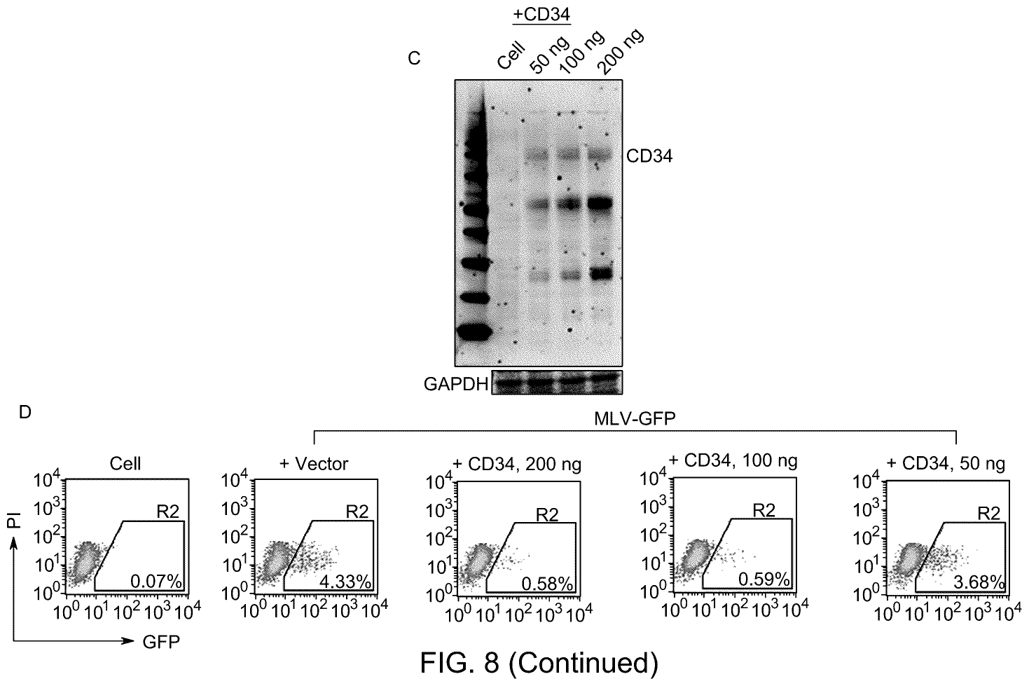

We further tested whether CD34 is also a broad-spectrum antiviral protein active against other retroviruses. We assembled an ectotropic murine leukemia reporter virus (MLV-GFP) in the presence of and absence of CD34 (FIG. 8). The infectivity of these particles was quantified in NIH3T3 target cells, and CD34 also inactivated MLV infectivity in a dosage-dependent manner (FIG. 8). Combined with previous demonstration of the anti-influenza A activity of CD34 (18), our studies suggest that CD34 has broad-spectrum antiviral activities against multiple enveloped viruses.

### INCORPORATION BY REFERENCES

All publications and patents referred are incorporated by reference in its entirety.
1. C. I. Civin et al., Antigenic analysis of hematopoiesis. III. A hematopoietic progenitor cell surface antigen defined by a monoclonal antibody raised against KG-la cells. J Immunol 133, 157-165 (1984).
2. R. W. Tindle et al., A novel monoclonal antibody BI-3C5 recognises myeloblasts and non-B non-T lymphoblasts in acute leukaemias and CGL blast crises, and reacts with immature cells in normal bone marrow. Leukemia Research 9, 1-9 (1985).
3. L. E. Sidney, M. J. Branch, S. E. Dunphy, H. S. Dua, A. Hopkinson, Concise review: evidence for CD34 as a common marker for diverse progenitors. Stem cells (Dayton, Ohio) 32, 1380-1389 (2014).
4. E. L. Berg, A. T. Mullowney, D. P. Andrew, J. E. Goldberg, E. C. Butcher, Complexity and differential expression of carbohydrate epitopes associated with L-selectin recognition of high endothelial venules. The American journal ofpathology 152, 469-477 (1998).
5. K. Suzawa et al., Preferential induction of peripheral lymph node addressin on high endothelial venule-like vessels in the active phase of ulcerative colitis. Am J Gastroenterol 102, 1499-1509 (2007).
6. J. S. Nielsen, K. M. McNagny, Novel functions of the CD34 family. J Cell Sci 121, 3683-3692 (2008).
7. M.-R. Blanchet et al., CD34 facilitates the development of allergic asthma. Blood 110, 2005-2012 (2007).
8. M.-R. Blanchet et al., CD34 is required for dendritic cell trafficking and pathology in murine hypersensitivity pneumonitis. Am J Respir Crit Care Med 184, 687-698 (2011).
9. E. Drew, J. S. Merzaban, W. Seo, H. J. Ziltener, K. M. McNagny, CD34 and CD43 Inhibit Mast Cell Adhesion and Are Required for Optimal Mast Cell Reconstitution. Immunity 22, 43-57 (2005).
10. B. Strilid et al., The Molecular Basis of Vascular Lumen Formation in the Developing Mouse Aorta. Developmental Cell 17, 505-515 (2009).
11. S. Baumheter et al., Binding of L-Selectin to the Vascular Sialomucin CD34. Science 262, 436-438 (1993).
12. D. B. AbuSamra et al., Not just a marker: CD34 on human hematopoietic stem/progenitor cells dominates vascular selectin binding along with CD44. Blood Advances 1, 2799-2816 (2017)
13. Y. Fu et al., PSGL-1 restricts HIV-1 infectivity by blocking virus particle attachment to target cells. Proc Natl Acad Sci USA 117, 9537-9545 (2020).
14. Y. Liu et al., Proteomic profiling of HIV-1 infection of human CD4(+) T cells identifies PSGL-1 as an HIV restriction factor. Nat Microbiol 4, 813-825 (2019).
15. T. Murakami, N. Carmona, A. Ono, Virion-incorporated PSGL-1 and CD43 inhibit both cell-free infection and transinfection of HIV-1 by preventing virus-cell binding. Proc Natl Acad Sci USA 117, 8055-8063 (2020).
16. Y. Wu, M. H. Beddall, J. W. Marsh, Rev-dependent indicator T cell line. Current HIV Research 5, 395-403 (2007).
17. Y. Wu, M. H. Beddall, J. W. Marsh, Rev-dependent lentiviral expression vector. Retrovirology 4, 12 (2007).
18. D. Dabbagh et al., Identification of the SHREK Family of Proteins as Broad-Spectrum Host Antiviral Factors. Viruses 13, 832 (2021).
19. V. Marechal, F. Clavel, J. M. Heard, O. Schwartz, Cytosolic Gag p24 as an index of productive entry of human immunodeficiency virus type 1. J Virol 72, 2208-2212 (1998).
20. G. G. Olinger, M. Saifuddin, G. T. Spear, CD4-Negative cells bind human immunodeficiency virus type 1 and efficiently transfer virus to T cells. J Virol 74, 8550-8557 (2000).
21. E. O. Freed, E. L. Delwart, G. L. Buchschacher, Jr., A. T. Panganiban, A mutation in the human immunodeficiency virus type 1 transmembrane glycoprotein gp41 dominantly interferes with fusion and infectivity. Proc Natl Acad Sci USA 89, 70-74. (1992).
22. R. J. McLinden et al., Detection of HIV-1 neutralizing antibodies in a human CD4(+)/CXCR4(+)/CCR5(+) T-lymphoblastoid cell assay system. PLoS One 8, e77756 (2013).
23. D. Yu, W. Wang, A. Yoder, M. Spear, Y. Wu, The HIV envelope but not VSV glycoprotein is capable of mediating HIV latent infection of resting CD4 T cells. PLoS Pathog 5, e1000633 (2009).
24. H. Asou et al., Establishment of an undifferentiated leukemia cell line (Kasumi-3) with t(3;7)(q27;q22) and activation of the EVI1 gene. Jpn J Cancer Res 87, 269-274 (1996).
25. C. C. Carter et al., HIV-1 infects multipotent progenitor cells causing cell death and establishing latent cellular reservoirs. Nat Med 16, 446-451 (2010).
26. F. Louache et al., Role of human immunodeficiency virus replication in defective in vitro growth of hematopoietic progenitors. Blood 80, 2991-2999 (1992).
27. F. F. Weichold et al., Neither Human Immunodeficiency Virus-1 (HIV-1) nor HIV-2 Infects Most-Primitive Human Hematopoietic Stem Cells as Assessed in Long-Term Bone Marrow Cultures. Blood 91, 907-915 (1998).
28. D. O. Griffin, S. P. Goff, K. L. Beemon, HIV-1 Is Restricted prior to Integration of Viral DNA in Primary Cord-Derived Human CD34<sup>+</sup> Cells. Journal of Virology 89, 8096-8100 (2015).
29. S. Kaushal et al., Exposure of human CD34+ cells to human immunodeficiency virus type 1 does not influence their expansion and proliferation of hematopoietic progenitors in vitro. Blood 88, 130-137 (1996).
30. C. M. Durand et al., HIV-1 DNA is detected in bone marrow populations containing CD4+ T cells but is not found in purified CD34+ hematopoietic progenitor cells in most patients on antiretroviral therapy. The Journal of infectious diseases 205, 1014-1018 (2012).
31. L. Josefsson et al., Hematopoietic precursor cells isolated from patients on long-term suppressive HIV therapy did not contain HIV-1 DNA. J Infect Dis 206, 28-34 (2012).
32. L. A. McNamara et al., CD133+ hematopoietic progenitor cells harbor HIV genomes in a subset of optimally treated people with long-term viral suppression. J Infect Dis 207, 1807-1816 (2013).
33. C. C. Nixon et al., HIV-1 infection of hematopoietic progenitor cells in vivo in humanized mice. Blood 122, 2195-2204 (2013).
34. T. D. Zaikos et al., Hematopoietic Stem and Progenitor Cells Are a Distinct HIV Reservoir that Contributes to Persistent Viremia in Suppressed Patients. Cell Reports 25, 3759-3773.e3759 (2018).
35. V. Bordoni et al., Bone marrow CD34+ progenitor cells may harbour HIV-DNA even in successfully treated patients. Clin Microbiol Infect 21, 290.e295-298 (2015).
36. T. M. Folks et al., Infection and Replication of HIV-1 in Purified Progenitor Cells of Normal Human Bone Marrow. Science 242, 919-922 (1988).
37. C. C. Carter et al., HIV-1 utilizes the CXCR4 chemokine receptor to infect multipotent hematopoietic stem and progenitor cells. Cell Host Microbe 9, 223-234 (2011).
38. M. Cavrois, C. De Noronha, W. C. Greene, A sensitive and specific enzyme-based assay detecting HIV-1 virion fusion in primary T lymphocytes. Nat Biotechnol 20, 1151-1154 (2002).

1. An in vitro method comprising:
co-expressing a viral vector of a virus of interest and a CD34 expression vector into a human cell line to produce virus particles such that CD34 incorporates into the virus particles;
isolating the virus particles from the human cell line;
blocking a viral infection; and wherein the method is configured for inhibiting the viral infection of the virus of interest.

2. The method of claim 1, wherein the virus particles comprise non-infectious virus particles, attenuated virus particles or inactivated virus particles of the virus of interest.

3. The method of claim 1, wherein the expression vector is in an amount of about 0.5 ng to about 400 ng.

4. The method of claim 1, wherein a ratio of the CD34 expression vector and the viral vector is about 1:1 to 1:1000.

5. The method of claim 1, wherein the CD34 expression vector inhibits the viral infection in a dose dependent manner.

6. The method of claim 1, wherein the virus of interest comprises HIV or MLV.

7. The method of claim 1, wherein the CD34 is configured to sterically hinder attachment of the virus particles to its corresponding target cells.

8. The method of claim 7, wherein the CD34 inhibits attachment of the virus particles to the target cells independent of an envelope glycoprotein.

9. (canceled)

10. The method of claim 1, wherein the method is configured to reduce the viral infection by about 25% to about 85%.

11. A composition comprising a recombinant virus particle comprising an overexpressed CD34 protein; wherein the recombinant virus particle comprises a non-infectious virus particle, an attenuated virus particle or an inactivated virus particle or a combination thereof.

12. (canceled)

13. The composition of claim 11, wherein the composition is configured to treat a viral infection.

14. The composition of claim 13, wherein the viral infection is caused by an enveloped virus.

15. (canceled)

16. The composition of claim 13, wherein the composition is configured to reduce the viral infection by about 25% to about 95%.

17-18. (canceled)

19. The composition of claim 11, wherein the composition comprises an adjuvant comprising an interleukin.

20. The composition of claim 14, wherein the enveloped virus comprises HIV, MLV or influenza.

21. An ex vivo method comprising:
taking CD34+ cells; transfecting a viral vector expressing a virus of interest into the CD34+ cells; producing virus particles in the CD34+ cells and isolating the virus particles from the CD34+ cells, and wherein the virus particles are non-infectious virus particles, attenuated virus particles or inactivated virus particles or a combination thereof of the virus of interest.

22. The method of claim 21, wherein the virus particles are configured to inhibit a viral infection of the virus of interest.

23. The method of claim 22, wherein the viral infection comprises HIV.

24-25. (canceled)

26. The method of claim 22, wherein the viral infection comprises influenza virus.

27. The method of claim 22, wherein the viral infection comprises MLV virus.

28-30. (canceled)
